(19) United States
(12) Patent Application Publication
(10) Pub. No.: US 2022/0313618 A1
(43) Pub. Date: Oct. 6, 2022

(54) PLANT VIRAL NUCLEIC ACID DELIVERY PARTICLES AND USES THEREOF

(71) Applicant: CASE WESTERN RESERVE UNIVERSITY, Cleveland, OH (US)

(72) Inventor: Nicole F. Steinmetz, San Diego, CA (US)

(21) Appl. No.: 17/837,325

(22) Filed: Jun. 10, 2022

Related U.S. Application Data
(63) Continuation-in-part of application No. 17/129,463, filed on Dec. 21, 2020.
(60) Provisional application No. 62/951,143, filed on Dec. 20, 2019.

(51) Int. Cl. A61K 9/51; C12N 15/113; A61K 38/19

(52) U.S. Cl. CPC A61K 9/5184; C12N 15/1135; A61K 38/193; C12N 2310/14; C12N 2320/32

(57) ABSTRACT

A nanoparticle includes a plant virus or plant virus like particle (VLP), an exogenous therapeutic nucleic acid encapsulated within the plant virus or plant VLP, and one or more fusogenic peptides or cell penetrating peptides conjugated to an exterior surface of the plant virus or plant VLP.

### RELATED APPLICATION

This application is a Continuation-in-Part of U.S. patent application Ser. No. 17/129,463, filed Dec. 21, 2020 which claims priority from U.S. Provisional Application No. 62/951,143, filed Dec. 20, 2019, the subject matter of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jun. 10, 2022, is named CWR028801USCIPSEQUENCELISTING.txt and is 7,119 bytes in size.

### TECHNICAL FIELD

This application relates to therapeutic nucleic acid-loaded plant virus particles and to their use in compositions for treating diseases and disorders in a subject.

### BACKGROUND

Nucleic acid delivery has long been recognized as a research tool in molecular biology to decipher the fundamental processes of life. In medicine, nucleic acid therapy is at the verge of becoming a clinical reality and nanomanufacturing schemes for the production of efficient and safe delivery vehicles are urgently needed. For example, small regulatory RNA therapeutics, such as siRNA, have wide ranging applications in the regulation of cell protein expression. Gene silencing with siRNA holds tremendous promise in cancer therapy and beyond; synthetic siRNAs can be designed to target in principle any gene of interest, therefore enabling downregulation of genes involved in cell proliferation, epithelial-mesenchymal transition, or drug resistance. However, to make a clinical impact, a delivery strategy is required, because ‘naked’ siRNA are not stable in plasma, not targeted, and their negative charge impairs cell uptake.

Proposed therapeutic nucleic acid delivery platforms have advantages and disadvantages. While mammalian viruses have been developed for gene therapy, these viruses have drawbacks such as possible adverse effects as a result of gene integration and their inherent immunogenicity. While non-viral systems generally offer safety, they do not match the effectiveness of viral delivery systems, as they can be instable in biological media leading to aggregation and/or premature cargo release. Therefore, there remains a continued need for the development of efficient delivery vehicles.

Plant-virus based-nanotechnologies provide an exciting alternative to the more traditional and more frequently exploited synthetic nanoparticles. Plant viruses, or viruses in general, can be considered as nature's delivery vehicles; viruses are designed to penetrate cells and deliver cargo. While mammalian viruses have been used to deliver genes for nucleic acid therapy, plant viruses offer a safer alternative due to their inability to infect or replicate in mammalian cells. Like other biologics, plant virus-based nanoparticles can be manufactured through a variety of homologous and heterologous expression systems at high yields and with high quality control and assurance. Plant viruses are monodisperse and many of their structures are known to near atomic resolution; therefore enabling structure-based design of high precision nanodrug delivery systems.

### SUMMARY

Embodiments described herein relate to nanoparticle constructs, plant virus vectors, pharmaceutical compositions comprising these nanoparticles or plant virus vectors, and methods for expressing a therapeutic nucleic acid in a mammal and for treating cancers in a subject using these plant virus vectors.

In some embodiments, a nanoparticle or plant virus vector includes a plant virus or plant virus like particle (VLP), an exogenous therapeutic nucleic acid encapsulated within the plant virus or plant VLP, and one or more fusogenic peptides or cell penetrating peptides conjugated to an exterior surface of the plant virus or plant VLP. By exogenous therapeutic nucleic acid it is meant an therapeutic peptide that is exogenous to or non-endogenous of the plant virus or plant VLP. The therapeutic nucleic acid can be capable of treating, ameliorating, attenuating, and/or eliminating symptoms of a disease or disorder in the subject when the composition is introduced to or within a cell or tissue of the subject with a disease or disorder

In some embodiments, the plant virus is a rod-shaped plant virus. In some embodiments, the plant virus is a tobacco mosaic virus. In some embodiments, the plant virus is an icosahedral-shaped plant virus. The icosahedral-shaped plant virus particle can belong to the Bromoviridae family. In some embodiments, the icosahedral-shaped plant virus particle is a cowpea chlorotic mottle virus (CCMV) virus particle.

In some embodiments, the therapeutic nucleic acid is capable of treating, ameliorating, attenuating, and/or eliminating symptoms of a disease or disorder in the subject when the composition is introduced to or within a cell or tissue of the subject with a disease or disorder.

In some embodiments, the therapeutic nucleic is an RNAi construct. The RNAi construct can be a siRNA, such as an siRNA targeting an oncogene. In some embodiments, the therapeutic nucleic acid can include a viral genome and a heterologous therapeutic gene. In some embodiments, the therapeutic nucleic acid includes an mRNA encoding a therapeutic protein.

In some embodiments, the plant virus particle or VLP includes a fusogenic peptide, cell penetrating peptide, or endolysosomal release agents. In some embodiments, the plant virus vector composition further includes one or more endolysosomal release agents. The fusogenic peptide, cell penetrating peptide, or endolysosomal release agents can be linked to the exterior surface of the plant virus particle. In some embodiments, the endolysosomal release agent can include an L17E M-lycotoxin peptide. In some embodiments, the plant virus particle or VLP includes stealth coating selected from the group consisting of PEG, albumin, and CD47 peptide.

Another embodiment relates to a method of expressing a therapeutic nucleic acid in a mammal. The method includes administering a plant virus vector composition to the mammal, the composition including a plant virus particle or virus-like particle (VLP) and a therapeutic nucleic acid, wherein the therapeutic nucleic acid is encapsulated within the plant virus particle or VLP. The composition can be administered to the subject systemically.

In some embodiments, the plant virus is a rod-shaped plant virus. In some embodiments, the plant virus is a tobacco mosaic virus. In some embodiments, the plant virus is an icosahedral-shaped plant virus. The icosahedral-shaped plant virus particle can belong to the Bromoviridae family. In some embodiments, the icosahedral-shaped plant virus particle is a cowpea chlorotic mottle virus (CCMV) virus particle.

In some embodiments, the therapeutic nucleic acid is capable of treating, ameliorating, attenuating, and/or eliminating symptoms of a disease or disorder in the subject when the composition is introduced to or within a cell or tissue of the subject with a disease or disorder.

In some embodiments, the therapeutic nucleic is an RNAi construct. The RNAi construct can be a siRNA, such as an siRNA targeting an oncogene. In some embodiments, the therapeutic nucleic acid can include a viral genome and a heterologous therapeutic gene. In some embodiments, the therapeutic nucleic acid includes an mRNA encoding a therapeutic protein.

In some embodiments, the plant virus particle or VLP includes a fusogenic peptide. In some embodiments, the plant virus vector composition further includes one or more endolysosomal release agents. The endolysosomal release agent can be linked to the exterior surface of the plant virus particle. In some embodiments, the endolysosomal release agent can include an L17E M-lycotoxin peptide. In some embodiments, the plant virus particle or VLP includes stealth coating selected from the group consisting of PEG, albumin, and CD47 peptide.

Another embodiment relates to a method of treating cancer in a subject. The method includes administering to the subject a therapeutically effective amount of a plant virus vector composition that includes a plant virus particle or VLP, a siRNA targeted an oncogene, wherein the siRNA is encapsulated within the plant virus particle or VLP. The nanoparticle can be administered to the subject systemically.

In some embodiments, the plant virus is a rod-shaped plant virus. In some embodiments, the plant virus is a tobacco mosaic virus. In some embodiments, the plant virus is an icosahedral-shaped plant virus. The icosahedral-shaped plant virus particle can belong to the Bromoviridae family. In some embodiments, the icosahedral-shaped plant virus particle is a cowpea chlorotic mottle virus (CCMV) virus particle.

In some embodiments, the therapeutic nucleic acid is capable of treating, ameliorating, attenuating, and/or eliminating symptoms of a disease or disorder in the subject when the composition is introduced to or within a cell or tissue of the subject with a disease or disorder.

In some embodiments, the therapeutic nucleic is an RNAi construct. The RNAi construct can be a siRNA, such as an siRNA targeting an oncogene. In some embodiments, the therapeutic nucleic acid can include a viral genome and a heterologous therapeutic gene. In some embodiments, the therapeutic nucleic acid includes an mRNA encoding a therapeutic protein.

In some embodiments, the plant virus particle or VLP includes a fusogenic peptide. In some embodiments, the plant virus vector composition further includes one or more endolysosomal release agents. The endolysosomal release agent can be linked to the exterior surface of the plant virus particle. In some embodiments, the endolysosomal release agent can include an L17E M-lycotoxin peptide. In some embodiments, the plant virus particle or VLP includes stealth coating selected from the group consisting of PEG, albumin, and CD47 peptide.

In some embodiments, the method can further include administering a therapeutically effective amount of an additional anticancer agent or therapy to the subject. The additional cancer agent can include an antitumor agent and/or an anti-hormonal agent. The additional anticancer therapy can include radiation therapy, brachytherapy, and/or ablation therapy.

### BRIEF DESCRIPTION OF THE FIGURES

FIGS. 1(A-D) are graphs and microscopic images showing (A) Flow cytometry was used to assess the uptake of CCMV-Cy5 in HeLa cells after incubation at 37° C. for 6 h. Following incubation, cells were treated with and without pronase to remove any loosely bound particles from the cell. (B) mean fluorescence intensity. (C, D) Confocal microscopy of HeLa cells (C), and HeLa cells with CCMV—Cy5 particles (D). Scale bar=25 μm.

FIGS. 2(A-F) are illustrations and images showing the characterization of reassembled CCMV particles. (A) Scheme for disassembly of whole CCMV virions to coat proteins, then the reassembly around heterologous siRNAs to make CCMV-siRNA. (B) Transmission electron micrograph of CCMV particles. (C) Transmission electron micrograph of reassembled CCMV particles. Scale bar=50 nm. (D) SDS-PAGE analysis of CCMV after conjugation with various molar excesses (600, 900, 1200:1 L17E:CCMV) of the cell penetrating peptide m-lycotoxin, L17E (CPP). The CCMV single coat protein is approximately 20 kDa. Successful conjugation is indicated by the higher molecular weight band (see arrow). (E) SDS-PAGE analysis of reassembled CCMV encapsulated eGFP siRNA or negative control siRNA. Lanes 1, 4=CCMV; 2, 5=reassembled CCMV; 3, 6=reassembled L17E-labeled CCMV. (F) Agarose gel electrophoresis showing successful encapsulation of siRNA in reassembled CCMV. Lane 1-CCMV (positive control); 2=CCMV-eGFPsiRNA; 3=CCMV-neg-siRNA.

FIGS. 3(A-G) are microscopic images and a graph showing siRNA silencing of HeLa/GFP cells. (A-F) Confocal microscopy of HeLA/GFP cells treated with different particle formulations for 24 hours. Loss of eGFP expression occurred when cells were treated with siRNA targeting eGFP. (A) HeLa/GFP cells only control. (B-C) Cells treated with CCMV-eGFPsiRNA and CCMV-mlyco-eGFPsiRNA (mlyco=L17E peptide), respectively. CCMV particles are presnt in cells with no eGFP expression. (D) Cells treated with lipofectamine+eGFPsiRNA; (E-F) with CCMV-negsiRNA and CCMV-mlyco-negsiRNA. Particles are visible in the cell indicating cell uptake, but no silencing of eGFP present. Scale bar =25 μm. (G) Quantitative real-time PCR showing relative levels of eGFP expression in cells after various treatments. Statistically significant changes in eGFP expression relative to the cells only control after a one-way ANOVA are indicated with *.

FIG. 4 is a graph showing quantitative real time PCR assessing the level of FOXA1 expression in MCF-7 cells after treatment with siRNAs, delivered with lipofectamine or encapsulated within CCMV and CCMV conjugated with m-lycotoxin L17E peptide (CPP). Statistically significant changes in FOXA1 expression relative to the cells only control after a one-way ANOVA are indicated with *.

FIG. 5 illustrates tobacco mosaic virus (left) and cowpea chlorotic mottle virus (right). For TMV the internal nucleic acid is a ssRNA genome. In the case of CCMV, the RNA cargo will beb loaded into the central cavity of the capsid (an RNA-free version is shown).

FIGS. 6(A-C) illustrate RNA-templated self-assembly (A) enables the production of full-length TMV (B, 300 nm in length) or lower-aspect ration version thereof (C, 60 nm in length). Any length can be programmed as a function of the length of the synthetic RNA transcript.

FIG. 7 illustrates synthetic gene design of a therapeutic nucleic acid in accordance with one embodiment.

FIG. 8 is a graphical illustration of size exclusion chromatography of intact TMV (black), its coat protein, and a 60 nm-sized reassembled VLP.

FIGS. 9 (A-D) illustrate A) Flow cytometry of CPMV and fusogenic CPMV-R5; R5=polyArg peptides at low (L) and high (H) density. The pronase treatment removes any surface bound nanoparticles. Fusogenic peptide coatings increase cell uptake and targeting of the cytoplasm (B-D). While CPMV is targeted to the endolysosome (as indicated by co-localization with Lamp-1 marker (C+D), fusogenic CPMV-R5 escapes and targets the cytoplasm (B+D) (in B the cell membrane was stained with wheat germ agglutinin, WGA). The nuclei are stained with DAPI.

FIG. 10 is a graphical illustration showing that while plant viral vectors also exhibit a protein corona, the corona formed was significantly less abundant compared to the corona formed on synthetic silica nanoparticles. TMV and spherical nanoparticles (SNP) formed from TMV coat proteins as well as silica noparticles were incubated in human plasma, then purified to study the protein corona. The complexes were analyzed by gel electrophoresis and the band analysis is shown indicating high abundance of plasma proteins bound to the synthetic silica nanoparticles (in black) and minimal protein adsorption on TMV-based nanoparticles.

FIGS. 11(A-F) illustrate several formulations of chemical ligation strategies for Sulfo-Cy5 (A) TMV and its coat protein (B). (C) Conjugation to internal glutamic acids using EDC coupling. (D) External modification of tyrosines via diazonium reaction or (E) lysines using NHS chemistry. (F) Click Chemistry.

FIG. 12 is a graphical illustration of interactions of TMV with macrophages as a function of TMV lengths (TS=60 nm, TM=130 nm, TL=300 nm), measured using flow cytometry.

FIG. 13(A-C) illustrate immune recognition of PEGylated and serum albumin (SA)-coated TMV. A, Immune recognition of fluorescent stealth TMV particles by α-TMV and α-SA antibodies dot blots. The binding of particles to α-TMV antibody spotted on the membrane is decreased by PEG coatings and effectively prevented by SA coating. B, Quantitative densitometric analysis of dot-blots (A) fluorescence signal. C, Schematic of the immunogenic properties of different TMV particles.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the application pertains. Commonly understood definitions of molecular biology terms can be found in, for example, Rieger et al., Glossary of Genetics: Classical and Molecular, 5th Edition, Springer-Verlag: New York, 1991, and Lewin, Genes V, Oxford University Press: New York, 1994.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, and other references mentioned herein are incorporated by reference in their entirety.

As used in the description of the invention and the appended claims, the singular forms “a,” “an,” and “the” are intended to include the plural forms as well, unless the context clearly indicates otherwise. In addition, the recitations of numerical ranges by endpoints include all numbers subsumed within that range (e.g., 1 to 5 includes 1, 1.5, 2, 2.75, 3, 3.80, 4, 5, etc.).

The terms “comprise,” “comprising,” “include,” “including,” “have,” and “having” are used in the inclusive, open sense, meaning that additional elements may be included. The terms “such as”, “e.g.,”, as used herein are non-limiting and are for illustrative purposes only. “Including” and “including but not limited to” are used interchangeably.

The term “or” as used herein should be understood to mean “and/or”, unless the context clearly indicates otherwise.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The term “nanoparticle” refers to any particle having a diameter of less than 1000 nanometers (nm). In general, the nanoparticles should have dimensions small enough to allow their uptake by eukaryotic cells. Typically, the nanoparticles have a longest straight dimension (e.g., diameter) of 200 nm or less. In some embodiments, the nanoparticles have a diameter of 100 nm or less. Smaller nanoparticles, e.g., having diameters of 50 nm or less, e.g., about 1 nm to about 30 nm or about 1 nm to about 5 nm, are used in some embodiments.

The phrases “parenteral administration” and “administered parenterally” are art-recognized terms and include modes of administration other than enteral and topical administration, such as injections, and include, without limitation, intratumoral, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrasternal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., tumor site), such that it enters the animal's system and, thus, is subject to metabolism and other like processes, for example, subcutaneous administration.

“Treating”, as used herein, means ameliorating the effects of, or delaying, halting or reversing the progress of a disease or disorder. The word encompasses reducing the severity of a symptom of a disease or disorder and/or the frequency of a symptom of a disease or disorder.

A “subject”, as used therein, can be a human or non-human animal. Non-human animals include, for example, livestock and pets, such as ovine, bovine, porcine, canine, feline and murine mammals, as well as reptiles, birds and fish. Preferably, the subject is human.

The language “effective amount” or “therapeutically effective amount” refers to a sufficient amount of the composition used in the practice of the invention that is effective to provide effective treatment in a subject, depending on the compound being used. That result can be reduction and/or alleviation of the signs, symptoms, or causes of a disease or disorder, or any other desired alteration of a biological system. An appropriate therapeutic amount in any individual case may be determined by one of ordinary skill in the art using routine experimentation.

A “prophylactic” or “preventive” treatment is a treatment administered to a subject who does not exhibit signs of a disease or disorder, or exhibits only early signs of the disease or disorder, for the purpose of decreasing the risk of developing pathology associated with the disease or disorder.

A “therapeutic” treatment is a treatment administered to a subject who exhibits signs of pathology of a disease or disorder for the purpose of diminishing or eliminating those signs.

“Pharmaceutically acceptable carrier” refers herein to a composition suitable for delivering an active pharmaceutical ingredient, such as the composition of the present invention, to a subject without excessive toxicity or other complications while maintaining the biological activity of the active pharmaceutical ingredient. Protein-stabilizing excipients, such as mannitol, sucrose, polysorbate-80 and phosphate buffers, are typically found in such carriers, although the carriers should not be construed as being limited only to these compounds.

The term “imaging agent” or “imaging moiety” can refer to a biological or chemical moiety capable being linked and/or conjugated directly or indirectly to nucleic acid loaded plant viral nanoparticles described herein and that may be used to detect, image, and/or monitor the presence and/or progression of a cell cycle, cell function/physiology, condition, pathological disorder and/or disease.

The term “polypeptide” or “peptide” is meant to refer to any polymer preferably consisting essentially of any of the 20 natural amino acids regardless of its size. Although the term “protein” is often used in reference to relatively large proteins, and “peptide” is often used in reference to small polypeptides, use of these terms in the field often overlaps. The term “polypeptide” refers generally to proteins, polypeptides, and peptides unless otherwise noted. Peptides described herein will be generally between about 0.1 to 100 KD or greater up to about 1000 KD, preferably between about 0.1, 0.2, 0.5, 1, 2, 5, 10, 20, 30 and 50 KD as judged by standard molecule sizing techniques such as centrifugation or SDS-polyacrylamide gel electrophoresis.

The terms “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

The term “nucleic acid” refers to oligonucleotides, nucleotides, polynucleotides, or to a fragment of any of these, to DNA or RNA (e.g., mRNA, rRNA, tRNA, miRNA, siRNA) of genomic or synthetic origin which may be single-stranded or double-stranded and may represent a sense or antisense strand, to peptide nucleic acids (PNAs), or to any DNA-like or RNA-like material, natural or synthetic in origin including, e.g., interfering RNA (iRNA), and ribonucleoproteins (e.g., iRNPs). The term can also encompass nucleic acids containing known analogues of natural nucleotides, as well as nucleic acid-like structures with synthetic backbones. Such categories of nucleic acids are well-known in the art.

The terms “inhibit,” “silencing,” and “attenuating” can refer to a measurable reduction in expression of a target mRNA (or the corresponding polypeptide or protein) as compared with the expression of the target mRNA (or the corresponding polypeptide or protein) in the absence of an interfering RNA molecule. The reduction in expression of the target mRNA (or the corresponding polypeptide or protein) is commonly referred to as “knock-down” and is reported relative to levels present following administration or expression of a non-targeting control RNA.

The term “iRNA agent,” as used herein, refers to small nucleic acid molecules used for RNA interference (RNAi), such as short interfering RNA (siRNA), double-stranded RNA (dsRNA), microRNA (miRNA) and short hairpin RNA (shRNA) molecules. The iRNA agents can be unmodified or chemically-modified nucleic acid molecules. The iRNA agents can be chemically synthesized or expressed from a vector or enzymatically synthesized. The use of a chemically-modified iRNA agent can improve one or more properties of an iRNA agent through increased resistance to degradation, increased specificity to target moieties, improved cellular uptake, and the like.

The term “antisense RNA,” as used herein, refers to a nucleotide sequence that comprises a sequence substantially complementary to the whole or a part of an mRNA molecule and is capable of binding to the mRNA.

The term “long non-coding ribonucleic acid”, “long non-coding RNA” or “lncRNA” refers to a ribonucleic acid sequence that is encoded within a genomic intronic or intergenic region. Such lncRNAs are not transcribed into proteins but act directly to regulate various activities including, but not limited to, transcription or translation. For example, an lncRNA may be exemplified by an aptamer that regulates transcription rates of a particular gene or allele.

The term “plasmid” is meant a circular nucleic acid vector. Plasmids contain an origin of replication that allows many copies of the plasmid to be produced in a bacterial or eukaryotic cell (e.g., 293T producer cell) without integration of the plasmid into the host cell DNA.

The term “gene” refers to a nucleic acid comprising a nucleotide sequence that encodes a polypeptide or a biologically active ribonucleic acid (RNA) such as a tRNA, shRNA, miRNA, etc. The nucleic acid can include regulatory elements (e.g., expression control sequences such as promoters, enhancers, an internal ribosome entry site (IRES)) and/or introns. A “gene product” or “expression product” of a gene is an RNA transcribed from the gene (e.g., pre- or post-processing) or a polypeptide encoded by an RNA transcribed from the gene (e.g., pre- or post-modification).

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of epithelial cells. The term also covers so-called “leaky” promoters, which regulate expression of a selected nucleic acid primarily in one tissue, but cause expression in other tissues as well.

The term “operably linked” refers to functional linkage between a nucleic acid expression control sequence (such as a promoter, or array of transcription factor binding sites) and a second nucleic acid sequence, wherein the expression control sequence directs transcription of the nucleic acid corresponding to the second sequence.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to plant virus vector compositions including a therapeutic nucleic acid-loaded plant virus nanoparticle. The nanoparticle includes a plant virus particle or virus like particle (VLP) and a therapeutic nucleic acid, wherein the therapeutic nucleic acid is encapsulated within the plant virus particle or VLP. The therapeutic nucleic acid can be loaded into the plant virus particle or VLP by noncovalently loading the nucleic acid within the plant virus particle or VLP capsid. The compositions described herein have numerous applications with respect to the delivery of nucleic acids to a subject. In some embodiments, the compositions described herein can be used in gene therapy to deliver therapeutic nucleic acid or genetic materials to cells and tissues of a subject in need thereof.

It has been shown using transmission electron microscopy (TEM) imaging that plant viral particles can be effectively noncovalently loaded with therapeutic nucleic acid, such as gene silencing siRNAs or mRNA encoding therapeutic proteins, thereby producing structurally sound nanoparticles capable of therapeutic nucleic acid delivery. For example, the icosahedral plant virus cowpea chlorotic mottle virus (CCMV) was shown to be effectively loaded with siRNAs targeting the forkhead box transcription factor (FOXA1) oncogene.

The plant virus vector compositions include plant virus particles and/or plant VLPs. Plant virus particles preferably grow in plants, have the advantages of being readily cultivated, and are unlikely to cause infection when used in vivo in a subject. The plant virus particles or VLPs can be nonreplicating and noninfectious when administered to a subject to avoid infection of the subject, and thus can be regarded as safe from a human health and agricultural perspective. In planta production prevents endotoxin contamination that may be a byproduct of other virus or VLP systems, for example those VLPs derived from E. coli. The plant virus particles or VLPs are scalable, stable over a range of temperatures (4-60° C.) and solvent:buffer mixtures.

In some embodiments, the plant virus particles or VLPs of the compositions can be based on icosahedral-shaped plant virus nanoparticles and/or icosahedral-shaped plant virus-like particles. An icosahedral-shaped plant virus is a small spherical virus that primarily infects plants, is non-enveloped and composed of capsid proteins that can self-assemble into well-organized icosahedral three-dimensional (3D) nanoscale multivalent architectures with high monodispersity and structural symmetry. Icosahedral-shaped plant viruses also include an exterior surface and interfaces between coat protein (CP) subunits that can be manipulated to allow for controlled self-assembly, and multivalent ligand display of nanoparticles or molecules for varied applications.

In some embodiments, the icosahedral-shaped plant virus belongs to a specific virus family, genus, or species. Examples of icosahedral-shaped plant viruses for use in a therapeutic nucleic acid-loaded plant virus nanoparticle described herein can be derived from the virus families Secoviridae, Geminiviridae, Luteoviridae, Bromoviridae, Phycodnaviridae, and Picornaviridae.

For example, in some embodiments, the icosahedral-shaped plant virus belongs to the Bromoviridae family. The Bromoviridae family includes the genus Bromovirus, Ilarvirus, Anulavirus, Oleavirus, and Cucumovirus. In some embodiments, the icosahedral-shaped plant virus belongs to the genus Bromovirus. The Bromovirus genus includes the species Brome mosaic virus (BMV), Broad Bean Mottle Virus (BBMV), Melandrium Yellow Fleck Virus (MYFV), Spring beauty latent virus (SBLV), Cassia yellow blotch virus (CYBV) and Cowpea Chlorotic Mottle Virus (CCMV).

In certain embodiments, the icosahedral-shaped plant virus belongs to the CCMV species. CCMV has a capsid constructed by 180 identical protein subunits each with a primary structure of 190 amino acid residues. There are three subunits are distributed over the virus coat, A, B, and C. The A subunits are arranged in pentamers and the B and C subunits are together arranged in hexamers. The virus coat is built up from 12 pentamers and 20 hexamers. Inside the capsid lies the (+)ssRNA genome consisting of around 3000 nucleotides.

In some embodiments, the icosahedral-shaped plant virus belongs to the Secoviridae family, which together with mammalian picornaviruses belong to the order of the Picornavirales. Secoviridae family plant viruses are relatively small having a diameter of about 30 nm, non-enveloped, positive-stranded RNA viruses with an icosahedral capsid. In some embodiments, the plant virus particles are selected from the Comovirinae virus subfamily of Secoviridae. Exemplary Comovirinae subfamily viruses for use in a method described herein can include Cowpea mosaic virus (CPMV), Broad bean wilt virus 1, and Tobacco ringspot virus. In certain embodiments, the plant virus or plant virus-like particles are from the genus Comovirus. A preferred example of a Comovirus is the CPMV or CPMV-like virus particles. The immune stimulating ability of CPMV is derived from its highly organized 3D protein architecture with its encapsulated nucleic acid and an intrinsic immune cell tropism. In some embodiments, the plant virus-like particle is an empty cowpea mosaic virus-like particle (eCPMV).

In one embodiment, CCMV can be propagated by mechanical inoculation using 5-10 μg of CCMV per leaf of cowpea plants, California Blackeye No. 5 (Vigna unguiculata). To isolate the virus, infected leaf material can be harvested 8 weeks post infection and purified. In another embodiment CPMV can be propagated in and purified from Vigna unguiculata plants with yields of 50-100 mg virus/100 g of infected leaves. In another embodiment, icosahedral plant virus, such as CCMV or CPMV, can be produced using an E. coli expression system.

In some embodiments, the plant virus or plant virus-like particle is a rod-shaped plant virus. A rod-shaped plant virus is a virus that primarily infects plants, is non-enveloped, and is shaped as a rigid helical rod with a helical symmetry. Rod shaped viruses also include a central canal. Rod-shaped plant virus particles are distinguished from filamentous plant virus particles as a result of being inflexible, shorter, and thicker in diameter. For example, Virgaviridae have a length of about 200 to about 400 nm, and a diameter of about 15-25 nm. Virgaviridae have other characteristics, such as having a single-stranded RNA positive sense genome with a 3′-tRNA like structure and no polyA tail, and coat proteins of 19-24 kilodaltons.

In some embodiments, the rod-shaped plant virus or virus-like particle belongs to a specific virus family, genus, or species. For example, in some embodiments, the rod-shaped plant virus belongs to the Virgaviridae family. The Virgaviridae family includes the genus Furovirus, Hordevirus, Pecluvirus, Pomovirus, Tobamovirus, and Tobravirus. In some embodiments, the rod-shaped plant virus belongs to the genus Tobamovirus. In further embodiments, the rod-shaped plant virus belongs to the tobacco mosaic virus (TMV) species. The tobacco mosaic virus has a capsid made from 2130 molecules of coat protein and one molecule of genomic single strand RNA 6400 bases long. The coat protein self-assembles into the rod like helical structure (16.3 proteins per helix turn) around the RNA which forms a hairpin loop structure. The protein monomer consists of 158 amino acids which are assembled into four main alpha-helices, which are joined by a prominent loop proximal to the axis of the virion. Virions are ˜300 nm in length and ˜18 nm in diameter. Negatively stained electron microphotographs show a distinct inner channel of ˜4 nm.

In other embodiments, the plant virus or plant virus-like particle is an Alphaflexiviridae virus or virus-like particle. The genera comprising the Alphaflexiviridae family include Allexivirus, Botrexvirus, Lolavirus, Mandarivirus, Potexvirus, and Sclerodarnavirus. In further embodiments, the plant virus particle of the vaccine composition is a Potexvirus particle. Examples of Potexvirus include Allium virus X, Alstroemeria virus X, Alternanthera mosaic virus, Asparagus virus 3, Bamboo mosaic virus, Cactus virus X, Cassava common mosaic virus, Cassava virus X, Clover yellow mosaic virus, Commelina virus X, Cymbidium mosaic virus, Daphne virus X, Foxtail mosaic virus, Hosta virus X, Hydrangea ringspot virus, Lagenaria mild mosaic virus, Lettuce virus X, Lily virus X, Malva mosaic virus, Mint virus X, Narcissus mosaic virus, Nerine virus X, Opuntia virus X, Papaya mosaic virus, Pepino mosaic virus, Phaius virus X, Plantago asiatica mosaic virus, Plantago severe mottle virus, Plantain virus X, Potato aucuba mosaic virus, Potato virus X, Schlumbergera virus X, Strawberry mild yellow edge virus, Tamus red mosaic virus, Tulip virus X, White clover mosaic virus, and Zygocactus virus X. In some embodiments, the plant virus-like particle is a Potato virus X virus-like particle.

The plant virus or plant virus-like particles for use in a composition described herein can be obtained according to various methods known to those skilled in the art. In embodiments where plant virus particles are used, the plant virus particles can be obtained from the extract of a plant infected by the plant virus. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant picornavirus particles using extraction of an infected plant are known to those skilled in the art. See Wellink J., Meth Mol Biol, 8, 205-209 (1998). Procedures are also available for obtaining virus-like particles. Saunders et al., Virology, 393(2):329-37 (2009). The disclosures of both of these references are incorporated herein by reference.

The therapeutic nucleic acid(s) that is loaded into and encapsulated within the interior of a plant virus particle of virus-like particle can include any nucleic acid that when introduced to or within cells and tissues of a subject is capable of treating, ameliorating, attenuating, and/or eliminating symptoms of a disease or disorder in the subject. The nucleic acid can be any nucleic acid encoding a natural, truncated, artificial, chimeric or recombinant product [e.g., a polypeptide of interest (including a protein or a peptide), a RNA, etc.] that is capable of treating, ameliorating, attenuating, and/or eliminating symptoms of a disease or disorder.

The nucleic acid can be a deoxyribonucleic acid (DNA) molecule (cDNA, gDNA, synthetic DNA, artificial DNA, recombinant DNA, etc.) or a ribonucleic acid (RNA) molecule (mRNA, tRNA, RNAi, siRNA, catalytic RNA, antisense RNA, viral RNA, etc.) or peptide nucleic acid (PNA). The nucleic acid may be single stranded or multistranded nucleic acid, double-stranded nucleic acid or may be complexed. The nucleic acid may comprise hybrid sequences or synthetic or semi-synthetic sequences. It may be obtained by any technique known to persons skilled in the art, and especially by screening libraries, by chemical synthesis, or alternatively by mixed methods including chemical or enzymatic modification of sequences obtained by screening libraries.

The nucleic acid loaded into and encapsulated within the interior of a plant virus particle or virus-like particle can include a nucleic acid from any source, such as a nucleic acid obtained from cells in which it occurs in nature, recombinantly produced nucleic acid, or chemically synthesized nucleic acid. For example, the nucleic acid can be cDNA or genomic DNA or DNA synthesized to have the nucleotide sequence corresponding to that of naturally-occurring DNA. The nucleic acid can also be a mutated or altered form of nucleic acid (e.g., DNA that differs from a naturally occurring DNA by an alteration, deletion, substitution or addition of at least one nucleic acid residue) or nucleic acid that does not occur in nature.

In some embodiments, the therapeutic nucleic acid can be a functional nucleic acid. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, siRNA, microRNA (miRNA), shRNA, lncRNA and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

In a particular embodiment, the therapeutic nucleic acid is of synthetic or biosynthetic origin or extracted from a virus or from a unicellular or pericellular eukaryotic or prokaryotic organism.

The therapeutic nucleic acid used may be naked, may be complexed with any chemical, biochemical or biological agent, may be inserted in a vector, etc., when loaded into the interior of a plant virus particle or VLP. The naked nucleic acid can refer to any nucleic acid molecule which is not combined with a synthetic, biosynthetic, chemical, biochemical or biological agent improving the delivery or transfer of said nucleic acid or facilitating its entry into a cell or cell compartment.

The vector can be a nucleic acid molecule that is capable of transporting another nucleic acid, such as a therapeutic nucleic acid, to which it has been linked. In some embodiments, the vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer to circular double stranded DNA loops which, in their vector form, are not bound to the chromosome. In some embodiments, the plasmid can be the most commonly used form of vector. In other embodiments, the plasmid can be a form of naked DNA as described herein.

In some embodiments, the nucleic acid may also contain one or more additional regions, for example regulatory elements of small or large size which are available to the skilled artisan, such as a promoter region (constitutive, regulated, inducible, tissue-specific, etc.), for example sequences allowing and/or promoting expression in a targeted tissue or cells, a transcription termination signal, secretion sequences, an origin of replication and/or nuclear localization signal (nls) sequences which further enhance polynucleotide transfer to the cell nucleus.

Additionally, the therapeutic nucleic acid may further comprise selectable markers useful in selecting, measuring, and monitoring nucleic acid transfer results (transfer to which tissues, duration of expression, etc.). The types of expression systems and reporter genes that can be used or adapted for use are well known in the art. For example, genes coding for a luciferase activity, an alkaline phosphatase activity, or a green fluorescent protein (GFP) activity are commonly used. In certain embodiments, the GFP can include EGFP, which is an engineered type of GFP for use in mammalian cells.

The therapeutic nucleic acid may contain any nucleotide sequence of any size. The nucleic acid may thus vary in size from a simple oligonucleotide to a larger molecule such as a nucleotide sequence including exons and/or introns and/or regulatory elements of any sizes (small or large), a gene of any size, for example of large size, or a chromosome for instance, and may be a plasmid, an episome, a viral genome, a phage, a yeast artificial chromosome, a minichromosome, an antisense molecule, etc.

In some embodiments, the therapeutic nucleic acid is a double-stranded, circular DNA, such as a plasmid, encoding a product with biological activity.

The therapeutic nucleic acid can be prepared and produced according to conventional recombinant nucleic acid techniques, such as amplification, culture in prokaryotic or eukaryotic host cells, purification, etc. The techniques of recombinant nucleic acid technology are known to those of ordinary skill in the art.

In some embodiments, the therapeutic nucleic acids include nucleic acids encoding therapeutic proteins or RNAs, including mRNAs and siRNAs. Such therapeutic nucleic acids may act by providing an activity that is missing, or significantly reduced, in a diseased cell or tissue. Such molecules may also act by modifying or reducing an activity that is over-expressed, or significantly elevated above normal levels, in a diseased cell or tissue. For example, a therapeutic nucleic acid may encode a protein possessing an activity (e.g., specific binding activity, enzymatic activity, transcriptional regulation activity, etc.) that is lacking in cells of a particular tissue. Lack of such activity may result from failure of the cells to produce the protein, production of a mutated, inactive form of the protein, or misfolding of a protein resulting in an inactive form. In some cases, introducing a “good” (i.e., functional) copy of the protein may alleviate symptoms of the disease by directly replacing the missing activity. Alternatively, therapeutic nucleic acids may act by increasing or decreasing the activity of other proteins in cells. For example, the therapeutic nucleic acid may encode a protein that may bind to another protein and thereby either decrease or eliminate the activity of the second protein. Alternatively, binding of the therapeutic nucleic acid may encode a protein that may bind to another protein in cells, which may result in stabilization of such protein and/or an increase in the related activity. Finally, the therapeutic nucleic acid may increase or decrease transcription of genes, or the translation of transcripts from genes in cells. For example, a therapeutic nucleic may encode a protein that may bind to a transcriptional region of a gene and thereby increase or decrease transcription of that gene.

The down regulation of gene expression using antisense nucleic acids can be achieved at the translational or transcriptional level. Antisense nucleic acids can be nucleic acid fragments capable of specifically hybridizing with a nucleic acid encoding an endogenous ocular active substance or the corresponding messenger RNA. These antisense nucleic acids can be synthetic oligonucleotides, optionally modified to improve their stability and selectivity. They can also be DNA sequences whose expression in the cell produces RNA complementary to all or part of the mRNA encoding an endogenous ocular active substance. Antisense nucleic acids can be prepared by expression of all or part of a nucleic acid encoding an endogenous ocular active substance, in the opposite orientation. Any length of antisense sequence is suitable for practice of the invention so long as it is capable of down-regulating or blocking expression of the endogenous ocular active substance. Preferably, the antisense sequence is at least 20 nucleotides in length. The preparation and use of antisense nucleic acids, DNA encoding antisense RNAs and the use of oligo and genetic antisense is disclosed in WO92/15680, the content of which is incorporated herein by reference.

In some embodiments, the nucleic acid encodes or is an RNAi or antisense polynucleotide sequences useful in eliminating or reducing the production of a gene product, as described by Tso, P. et al Annals New York Acad. Sci. 570:220-241 (1987). “RNA interference” or “RNAi” is a term initially applied to a phenomenon observed in plants and worms where double-stranded RNA (dsRNA) blocks gene expression in a specific and post-transcriptional manner. As used herein, the term “dsRNA” refers to siRNA molecules or other RNA molecules including a double stranded feature and able to be processed to siRNA in cells, such as hairpin RNA moieties. In certain embodiments, RNAi include RNAi that decrease the level of an apoptotic or angiogenic factor in a cell.

In some embodiments, an RNAi construct is loaded into and encapsulated within the interior of a plant virus particle or virus-like particle. As used herein, the term “RNAi construct” is a generic term used throughout the specification to include small interfering RNAs (siRNAs), hairpin RNAs, and other RNA species, which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

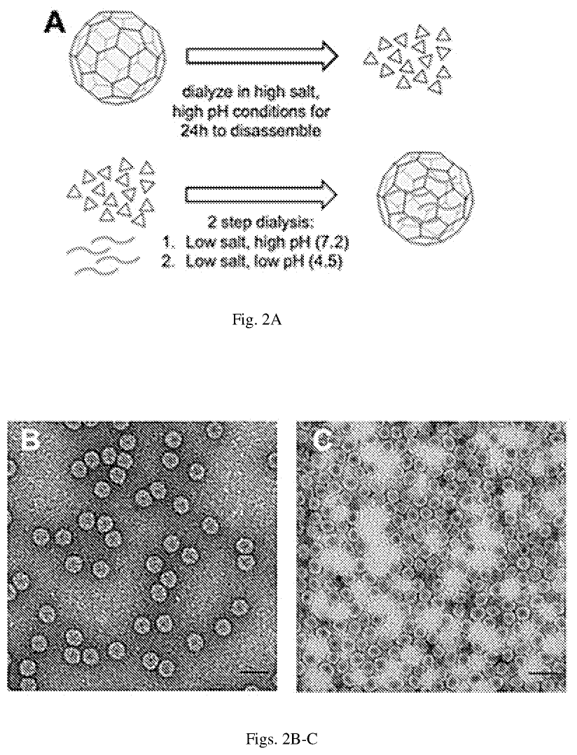

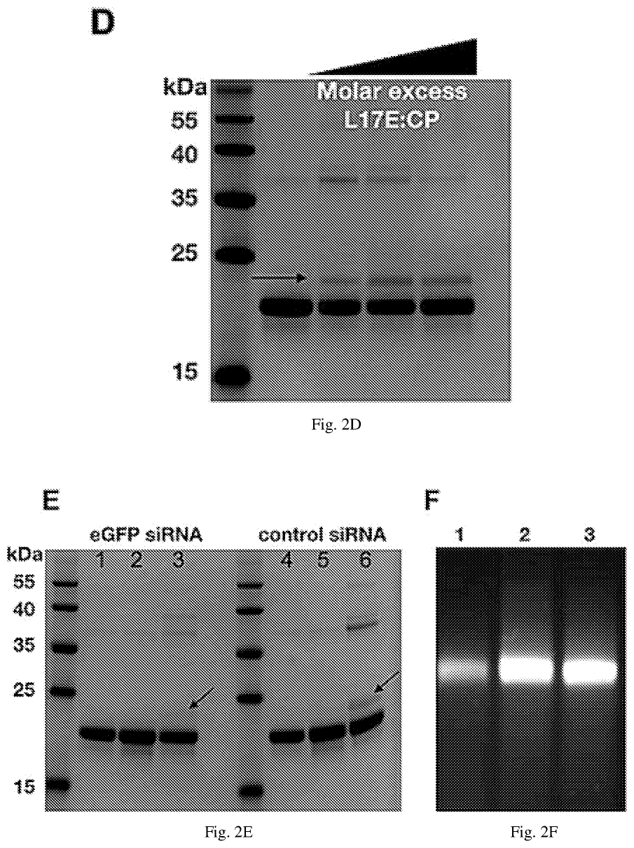

In an exemplary embodiment, an RNAi construct is loaded into and encapsulated within a CCMV capsid using pH- and salt-controlled, dis- and assembly methods to yield CCMV loaded with siRNA targeting the oncogene FOXA1, where the siRNA are added at a 6:1 (w/w) ratio (see FIG. 2A) to form about 30 nm-sized icosahedral particles (FIG. 2B, C). In certain embodiments, about 2-3 μM siRNAs can be encapsulated by the plant virus particle or virus-like particle.

“RNAi expression vector” (also referred to herein as a “dsRNA-encoding plasmid”) refers to replicable nucleic acid constructs used to express (transcribe) RNA which produces RNAi moieties, such as siRNA moieties, in the cell in which the construct is expressed. Such vectors include a transcriptional unit comprising an assembly of (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences.

The term “loss-of-function,” as it refers to genes inhibited by the subject therapeutic nucleic acids, refers to a decrease or diminishment in the level of expression of a gene when compared to the level in the absence of a therapeutic nucleic acid, such as RNAi constructs described herein. The term “expression”, as used here, means the overall flow of information from a gene to produce a gene product (typically a protein, optionally post-translationally modified or a functional/structural RNA).

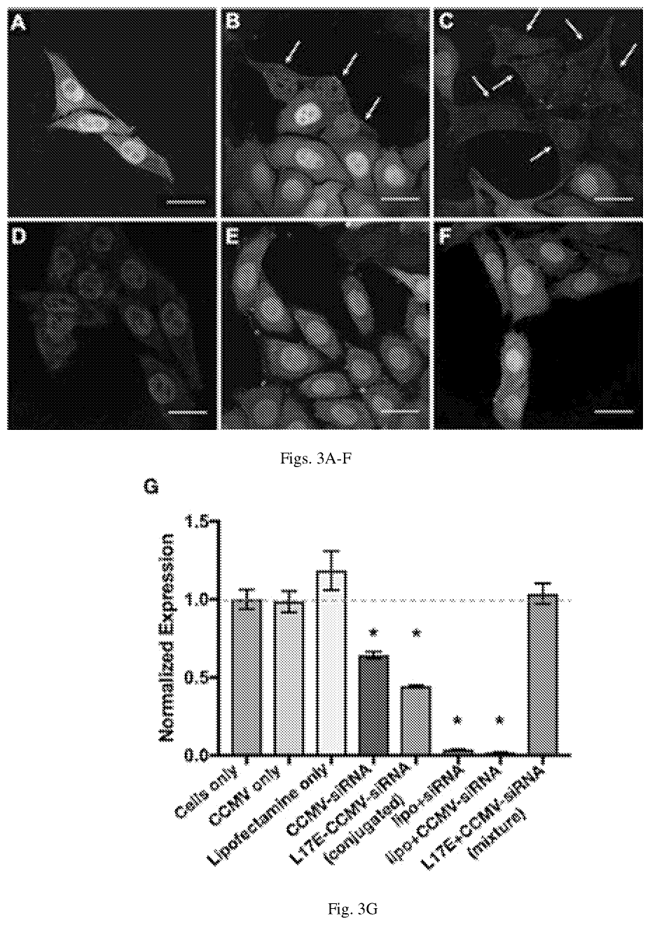

In some embodiments, the RNAi constructs loaded into a plant virus particle or virus like particle can decrease the expression level of a therapeutic target in a cell of a subject in need thereof using gene silencing. For example, it was shown using confocal microscopy successful gene silencing mediated by an icosahedral-shaped plant viral siRNA delivery vector described herein (see FIG. 3A-F).

In some embodiments, an RNAi construct reduces the level of a polynucleotide product that induces or promotes apoptosis in a cell. Genes whose polynucleotide products induce or promote apoptosis are referred to herein as “pro-apoptotic genes” and the products of those genes (mRNA; protein) are referred to as “pro-apoptotic polynucleotide products.” Pro-apoptotic polynucleotide products include, e.g., Bax, Bid, Bak, and Bad polynucleotide products. See, e.g., U.S. Pat. No. 7,846,730. Interfering RNAs could also be against an angiogenic product, for example VEGF (e.g., Cand5; see, e.g., U.S. Patent Publication No. 2011/0143400; U.S. Patent Publication No. 2008/0188437; and Reich et al. (2003) Mol. Vis. 9:210), VEGFR1 (e.g., Sirna-027; see, e.g., Kaiser et al. (2010) Am. J. Ophthalmol. 150:33; and Shen et al. (2006) Gene Ther. 13:225), or VEGFR2 (Kou et al. (2005) Biochem. 44: 15064). See also, U.S. Pat. Nos. 6,649,596, 6,399,586, 5,661,135, 5,639,872, and 5,639,736; and 7,947,659 and 7,919,473.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

Thus, embodiments tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript.

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of a nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, a modified ribonucleotide can be introduced by in vitro enzymatic or organic synthesis.

Methods of chemically modifying RNA molecules can be adapted for modifying RNAi constructs (see for example, Nucleic Acids Res, 25:776-780; J Mol Recog 7:89-98; Nucleic Acids Res 23:2661-2668; Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodie-sters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount, which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the therapeutic nucleic acid RNAi constructs can include “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

siRNAs specifically incorporate into the RNA-induced silencing complex (RISC) and then guide the RNAi machinery to destroy the target mRNA containing the complementary sequences. Since RNAi is based on nucleotide base-pairing interactions, it can be tailored to target any gene of interest, rendering siRNA an ideal tool for treating diseases with gene silencing. Gene silencing with siRNAs has a great potential for the treatment of human diseases as a new therapeutic modality. Numerous siRNAs have been designed and reported for various therapeutic purposes and some of the siRNAs have demonstrated specific and effective silencing of genes related to human diseases and disorders. Therapeutic applications of siRNAs include, but are not limited to, inhibition of viral gene expression and replication in antiviral therapy, anti-angiogenic therapy, treatment of autoimmune diseases and neurological disorders, and anticancer therapy. Therapeutic gene silencing has been demonstrated in mammals, which bodes well for the clinical application of siRNA. It is believed that siRNA can target every gene in human genome and has unlimited potential to treat human disease with RNAi.

The siRNA molecules described herein can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Proc Natl Acad Sci USA, 98:9742-9747; EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer to produce a dicer-substrate siRNA. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides, also referred to as 21 to 23-mer designs as the length of the oligonucleotide is usually denoted by “-mer” (from Greek meros, “part”).

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In some embodiments, the therapeutic nucleic acid is a RNAi construct in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Genes Dev, 2002, 16:948-58; Nature, 2002, 418:38-9; RNA, 2002, 8:842-50; and Proc Natl Acad Sci, 2002, 99:6047-52. Preferably, such hairpin RNAs are engineered in cells or in an animal to ensure continuous and stable suppression of a desired gene. It is known in the art that siRNAs can be produced by processing a hairpin RNA in the cell.

In some embodiments, the nucleic acid can encode or be an RNAi construct, such as siRNA, that inhibits the expression of long noncoding RNA (lncRNA), such as onco-lncRNA. LncRNAs are the largest class of noncoding RNAs, comprising over 20,000 genes annotated in the ENcylopedia of DNA Elements (ENCODE) and other reference databases. They are defined as transcripts produced by RNA polymerase II that are longer than 200 nucleotides and devoid of an open reading frame that can be translated into a protein. Derrien et al., 2012. “The GENCODE v7 catalog of human long noncoding RNAs: analysis of their gene structure, evolution, and expression. Genome Res 22: 1775-1789.

The function of most lncRNAs is unknown, but they have been proposed to play roles in both negatively and positively regulating gene expression. They can regulate expression of protein-coding genes at both the transcriptional and posttranscriptional levels. Posttranscriptional regulation could occur by competing with endogenous RNA to regulate microRNA levels, modulating mRNA stability and translation by homologous base pairing, or altering cellular localization of mRNAs. Transcriptional regulation can occur in cis with their effects restricted to the chromosome from which they are transcribed and in trans with their effects targeting gene transcription on other chromosomes. Both cis- and trans-acting lncRNAs can mediate their effects through their RNA transcripts; cis-acting lncRNAs can also regulate gene transcription as a result of the process of splicing or of transcription itself.

LncRNAs have been implicated in the regulation of tissue and developmental stage-specific transcription of genes and mutations and dysregulation of lncRNAs have been increasingly linked with diverse human diseases including cancer. LncRNAs have recently been identified in the development of multiple oncogenic processes, including EMT, inflammation, cancer stemness, metastasis, and drug-resistance. Aberrant overexpression of oncogenic lncRNAs (onco-lncRNAs) is disease- and tissue-specific and can influence global gene signatures and clinical outcomes by regulating multi-level gene expression. Therefore, in some embodiments, the nucleic acid can be an RNAi nucleic acid, such as siRNA, that inhibits the expression of onco-lncRNA.

In another embodiment, a plant virus particle or virus-like particle can be loaded with gene silencing antisense oligonucleotides (ASOs) that reduce or inhibit expression of a target gene. Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

The binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their delivery to a cell through encapsulation in an icosahedral-shaped plant virus particle or virus-like particle allows the degradation of the mRNA encoding a specific protein. Accordingly, antisense oligonucleotides decrease the expression and/or activity of a particular protein (e.g., an oncoprotein).

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc.

Oligonucleotides described herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (Nucl. Acids Res. 16:3209), methylphosphonate oligonucleotides can be prepared by use of controlled pore glass polymer supports (Proc Natl Acad Sci 85:7448-7451).

The selection of an appropriate oligonucleotide can be performed by one of skill in the art. Given the nucleic acid sequence encoding a particular protein, one of skill in the art can design antisense oligonucleotides that bind to that protein, and test these oligonucleotides in an in vitro or in vivo system to confirm that they bind to and mediate the degradation of the mRNA encoding the particular protein. To design an antisense oligonucleotide that specifically binds to and mediates the degradation of a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide and compare the sequence of that oligonucleotide to nucleic acid sequences that are deposited in publicly available databases to confirm that the sequence is specific or substantially specific for a particular protein.

In some embodiments, the therapeutic nucleic acid loaded into and encapsulated within a plant virus particle or virus-like particle can be can encode or be a miRNA (microRNA) nucleic acid. The term “miRNA” is used according to its ordinary and plain meaning and refers to a microRNA molecule found in eukaryotes that is involved in RNA-based gene regulation. The term can be used to refer to the single-stranded RNA molecule processed from a precursor or in certain instances the precursor itself. miRNAs are short non-coding RNAs, about 21-25 nucleotides in length, that regulate gene expression post-transcriptionally. miRNAs generally bind to the 3′-UTR (untranslated region) of their target mRNAs and repress protein production by destabilizing the mRNA and translational silencing. Over 2000 miRNAs that have been discovered in humans and it is believed that they collectively regulate one third of the genes in the genome. Numerous miRNAs have been linked to many human diseases and have been identified as therapeutic targets.

In some embodiments, the therapeutic nucleic acid loaded into and encapsulated within the interior of a plant virus particle of virus-like particle can encode biologically active polypeptides or proteins including enzymes, blood derivatives, hormones, lymphokines, cytokines, chemokines, anti-inflammatory factors, growth factors, trophic factors, neurotrophic factors, haematopoietic factors, angiogenic factors, anti-angiogenic factors, inhibitors of metalloproteinase, regulators of apoptosis, coagulation factors, receptors thereof, in particular soluble receptors, a peptide which is an agonist or antagonist of a receptor or of an adhesion protein, antigens, antibodies, fragments or derivatives thereof and other essential constituents of the cell. In some embodiments, the biologically active nucleic acid encodes a precursor of a therapeutic polypeptide or protein such as those described above.

In some embodiments, the therapeutic nucleic acid may encode for a viable protein so as to replace the defective protein which is naturally expressed in the targeted tissue. In other embodiments, the therapeutic nucleic acid encodes a site-specific endonuclease that provides for site-specific knock-down of gene function, e.g., where the endonuclease knocks out an allele associated with a particular disease or disorder. In addition to knocking out a defective allele, a site-specific nuclease can also be used to stimulate homologous recombination with a donor DNA that encodes a functional copy of the protein encoded by the defective allele. Thus, e.g., the compositions and methods described herein can be used to deliver both a site-specific endonuclease that knocks out a defective allele, and can be used to deliver a functional copy of the defective allele, resulting in repair of the defective allele, thereby providing for production of a functional protein. See, e.g., Li et al. (2011) Nature 475:217.

Site-specific endonucleases encoded by a therapeutic nucleic acid that are suitable for use include, e.g., zinc finger nucleases (ZFNs); and transcription activator-like effector nucleases (TALENs), where such site-specific endonucleases are non-naturally occurring and are modified to target a specific gene. Such site-specific nucleases can be engineered to cut specific locations within a genome, and non-homologous end joining can then repair the break while inserting or deleting several nucleotides. Such site-specific endonucleases (also referred to as “INDELs”) then throw the protein out of frame and effectively knock out the gene. See, e.g., U.S. Patent Publication No. 2011/0301073. Nucleases can be screened for activity prior to use, for example in a yeast-based chromosomal system as described in U.S. Pat. No. 8,563,314.

In some embodiments, the therapeutic nucleic acid can include nucleic acid encoding CRISPR/Cas nuclease system nucleases. See, e.g., U.S. Pat. No. 8,697,359 and U.S. patent application Ser. No. 14/278,903. The CRISPR (clustered regularly interspaced short palindromic repeats) locus, which encodes RNA components of the system, and the cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of CRISPR-associated (Cas) genes as well as non-coding RNA elements capable of programming the specificity of the CRISPR-mediated nucleic acid cleavage. CRISPR-Cas systems are separated into two classes. Class 1 uses several Cas proteins together with the CRISPR RNAs (crRNA) to build a functional endonuclease. Class 2 CRISPR systems use a single Cas protein with a crRNA.

The Cas9 related CRISPR/Cas system comprises two RNA non-coding components: tracrRNA and a pre-crRNA array containing nuclease guide sequences (spacers) interspaced by identical direct repeats (DRs). To use a CRISPR/Cas system to accomplish genome engineering, both functions of these RNAs must be present (see Cong et al, (2013) Sciencexpress1/10.1126/science 1231143). In some embodiments, the tracrRNA and pre-crRNAs are supplied via separate expression constructs or as separate RNAs.

In other embodiments, a chimeric RNA is constructed where an engineered mature crRNA (conferring target specificity) is fused to a tracrRNA (supplying interaction with the Cas9) to create a chimeric cr-RNA-tracrRNA hybrid (also termed a single guide RNA or sgRNA). In other embodiments, the nucleic acid encodes CRISPR/Cas Cpfl system nucleases. Cpfl cleaves DNA in a staggered pattern and requires only one RNA rather than the two (tracrRNA and crRNA) needed by Cas9 for cleavage.

In some embodiments, therapeutic nucleic acids can include nucleic acid encoding or a small gene fragment that encodes dominant-acting synthetic genetic elements (SGEs), e.g., molecules that interfere with the function of genes from which they are derived (antagonists) or that are dominant constitutively active fragments (agonists) of such genes. SGEs can include, but are not limited to, polypeptides, inhibitory antisense RNA molecules, ribozymes, nucleic acid decoys, and small peptides. The small gene fragments and SGE libraries disclosed in U.S. Patent Publication No. 2003/0228601, which is incorporated by reference, can be used herein.

In some embodiments, the therapeutic nucleic acid can include a nucleic acid encoding a ribozyme. Ribozymes, also called catalytic RNA, are RNA enzymes that catalyze a catalyze specific reactions similar to protein enzymes. Ribozymes are known to play a role in forming protein chains, RNA splicing, transfer RNA biosynthesis, and viral replication.

Therapeutic nucleic acids useful herein can also encode for immunity-conferring polypeptides, which can act as endogenous immunogens to provoke a humoral or cellular response, or both. The nucleic acids or polynucleotides employed can also code for an antibody. In this regard, the term “antibody” encompasses whole immunoglobulin of any class, chimeric antibodies and hybrid antibodies with dual or multiple antigen or epitope specificities, and fragments, such as F(ab)2, Fab2, Fab and the like, including hybrid fragments. Also included within the meaning of “antibody” are conjugates of such fragments, and so-called antigen binding proteins (single chain antibodies) as described, for example, in U.S. Pat. No. 4,704,692, the contents of which are hereby incorporated by reference.

A therapeutic nucleic acid can also include a synthetic immunostimulatory nucleic acid sequence. Synthetic immunostimulatory nucleic acids such as CpG DNA are being harnessed therapeutically as vaccine adjuvants, anticancer or antiallergic agents. CpG stimulatory motifs are reported to play an important role in the immunogenicity of nucleic acid vaccines. The innate immune system recognizes specific sequence motifs expressed in the DNA and/or RNA of various pathogens. For example, recognition of CpG motifs in DNA or dsRNA molecules triggers a strong proinflammatory response via toll like receptors (TLRs).

CpG oligodeoxynucleotides (or CpG ODN) are short single-stranded synthetic DNA molecules that contain a cytosine triphosphate deoxynucleotide (“C”) followed by a guanine triphosphate deoxynucleotide (“G”). The “p” refers to the phosphodiester link between consecutive nucleotides, although some ODN have a modified phosphorothioate (PS) backbone instead. When these CpG motifs are unmethylated, they act as immunostimulants. CpG motifs are considered pathogen-associated molecular patterns (PAMPs) due to their abundance in microbial genomes but their rarity in vertebrate genomes. The CpG PAMP is recognized by the pattern recognition receptor (PRR) Toll-Like Receptor 9 (TLR9), which is constitutively expressed only in B cells and plasmacytoid dendritic cells (pDCs) in humans and other higher primates.

In some embodiments, the therapeutic nucleic acid can include a nucleic acid sequence encoding a vaccine, such as a DNA or mRNA vaccine. Plasmid DNA and mRNA vaccines can be used for a variety of applications ranging from prophylaxis to therapy and from personalized medicine to global health solutions. DNA and mRNA vaccines can be readily made using well known generic manufacturing processes and can be constructed directly from the genetic sequence of the desired protein, whether the origin of the protein is human or from a pathogen. For vaccines, making a gene construct coding for the antigen instead of inactivating or attenuating the pathogen, or instead of making a recombinant protein, is easier, more rapid, and avoids potential risks of working with live pathogens. Likewise, the vaccine nucleic acid construct can encode only the key antigen without including other proteins that may be either deleterious (such as toxins) or that may be irrelevant for protection yet immunodominant.

Following endocytosis, therapeutic nucleic acid loaded plant virus particle compositions can become entrapped in an intracellular vesicle which delivers the particles to an endosome. In order to avoid nucleic acid degradation by acid hydrolases and nucleases, which would occur in the endosome after fusion with a lysosome, the nucleic acid must escape from the endosome before lysosomal fusion. Thus, in some embodiments, the plant virus vector compositions described herein can be designed so that the resulting nucleic acid loaded plant virus particle or VLP escapes endosomal and/or lysosomal compartments at the endosomal-lysosomal pH. For example, the plant virus or VLP loaded with therapeutic nucleic acid can be designed such that its structure and amphiphilicity changes at endosomal-lysosomal pH (5.0-6.0) and disrupts endosomal-lysosomal membranes, which allows entry of the plant virus nanoparticle into the cytoplasm. In one embodiment, the ability of specific endosomal-lysosomal membrane disruption of the compositions described herein can be tuned by modifying their pH sensitive amphiphlicity by altering the number and structure of protonatable amines and lipophilic groups. For example, decreasing the number of protonatable amino groups can reduce the amphiphilicity of a nanoparticle produced by the compound at neutral pH. In one aspect, the compounds herein have 1 to 50, 1 to 40, 1 to 30, 1 to 20, 1 to 10, 1 to 8, 1 to 6, 1 to 4, or 2 protonatable amino or substituted amino groups. The pH-sensitive amphiphilicity of the compounds and nanoparticles produced by the compounds can be used to fine-tune the overall pKa of the nanoparticle. Low amphiphilicity of the nanoparticles at physiological pH can minimize non-specific cell membrane disruption and nonspecific tissue uptake of the nucleic acid. In certain embodiments, it is desirable that the carriers have low amphiphilicity at the physiological pH and high amphiphilicity at the endosomal-lysosomal pH, which will only cause selective endosomal-lysosomal membrane disruption with the nanoparticles.

In some embodiments, a plant viral particle or VLP loaded with a therapeutic nucleic acid can be appended with or conjugated to one or more fusogenic peptide, cell penetrating peptide, or endolysosomal release agents to facilitate protein expression in cells by disrupting endolysosomal membranes. The terms fusogenic peptide, cell penetrating peptide, or endolysosomal release agent are used interchangeably herein and can include any agent capable of enhancing cell uptake and intracellular trafficking. In some embodiments, the endolysosomal release agent, can facilitate endolysosomal release of therapeutic nucleic acid entrapped in the endolysosomal compartment into the cytoplasm of a targeted cell of the subject, thereby overcoming the need for co-delivery of a transfection agent, such as Lipofectamine.

In some embodiments, the endolysosomal release agent can include a fusogenic peptide conjugated to the plant virus particle or VLP capsid to facilitate endosomal escape. In an exemplary embodiment, the fusogenic peptide includes a synthetic peptide containing the 20-aminoterminal amino acid sequence of influenza virus hemagglutinin(HA), where the peptide is stable at pH 7 but the acidic interior of the endosome elicits fusion with the endosomal membrane allowing nucleic acid particles to be released into the cytosol.

In some embodiments, the one or more endolysosomal release agents can include a cell penetrating peptide (CPP), also known as a protein transduction domain (PTD). In addition to enhancing endolysosomal release, CPPs for use in a composition described herein can facilitate uptake of the plant virus or virus like particle into a cell where the loaded nucleic acid can provide efficient gene silencing in targeted cells.

The number of endolysosomal release agents appended to a plant virus particle or VLP may affect (e.g., increase) the endolysosomal release and/or uptake of the nanoparticle by a desired cell. In some embodiments, a plant virus particle or VLP loaded with a therapeutic nucleic acid as described herein, such as a RNAi construct, can display about 10 to about 100 cell penetrating peptides per particle. In exemplary embodiments, an icosahedral-shaped plant virus particle or VLP loaded with an RNAi construct can display about 30 endolysosomal release agents per particle.

Endolysosomal release agents can be coupled to a therapeutic nucleic acid loaded plant virus particle or VLP either directly or indirectly (e.g., via a linker group). In some embodiments, the endolysosomal release agents can be covalently conjugated to coat proteins of the plant virus particle or VLP. The covalent link can include a peptide bond or a labile bond (e.g., a bond readily cleavable or subject to chemical change in the interior target cell environment).

In some embodiments, an endolysosomal release agent is conjugated to a coat protein of a plant virus particle or VLP via a linker. In an exemplary embodiment, peptide endolysosomal release agents can be synthesized with a C-terminal amide or Gly-Gly-Cys linker allowing for conjugation of the peptide endolysosomal release agent to surface lysines of a CCMV virus particle using an SM(PEG)4 linker.

In some embodiments, CPPs can include at least one transport peptide sequence that allows the plant virus particle or VLP to penetrate into a cell, such as a cancer cell. Examples of transport sequences that can be used in accordance with the present invention include a TAT-mediated protein delivery sequence (GRKKRRQRRRPQ) (SEQ ID NO: 1) (Vives (1997) 272: 16010-16017), polyargine sequences (Wender et al. 2000, PNAS 24: 13003-13008) and antennapedia (Derossi (1996) J. Biol. Chem. 271: 18188-18193). Other examples of known transport peptide moieties, subdomains and the like are described in, for example, Canadian patent document No. 2,301,157 (conjugates containing homeodomain of antennapedia) as well as in U.S. Pat. Nos. 5,652,122, 5,670,617, 5,674,980, 5,747,641, and 5,804,604, all of which are incorporated herein by reference in their entirety. Such transport moieties include conjugates containing amino acids of Tat HIV protein; herpes simplex virus-1 DNA binding protein VP22, a Histidine tag ranging in length from 4 to 30 histidine repeats, or a variation derivative or homologue thereof capable of facilitating uptake of the active cargo moiety by a receptor independent process.

CPPs are short peptides (<30 amino acids long) that are able to penetrate biological membranes and drive the internalization of a bioactive cargo in cells. CPPs for use in a can include positively and negatively charged, amphipathic (primary or secondary) and non-amphipathic CPPs. CPPs can be placed into the following three main groups: PTDs (Tat, Penetratin, etc.); model peptides (R9, KLAK); and designed peptides (Pep-1, sequence: KETWWETWWTEWSQPKKKRKV) (SEQ ID NO: 2). A review of cell-penetrating peptides can be found in Kalafatovic and Giralt, Molecules, 22(11), 1929 (2017) incorporated by reference in its entirety.

Penetratin (RQIKIWFQNRRMKWKK) (SEQ ID NO: 3) is a CPP, of which the first 16 amino acids are derived from the third alpha helix of the Antennapedia protein. Penetratin has been shown to enable proteins (made as fusion proteins) to cross cellular membranes (PCT international publication number WO 99/11809, incorporated by reference in its entirety). Similarly, HIV Tat protein was shown to be able to cross cellular membranes (Frankel A. D. et al., Cell, 55: 1189).

In some embodiments, the endolysosomal release agent can include endosomolytic peptide CPPs derived from the cationic and membrane-lytic spider venom peptide M-lycotoxin (see Akishiba et al. Nature Chemistry, 9, 751-761 (2017)). These delivery peptides were developed by introducing one or two glutamic acid residues into the hydrophobic face. In a particular embodiment, the transport moiety includes the M-lycotoxin peptide having the substitution of leucine by glutamic acid (L17E). L17E has been shown to promote cell uptake by micropinocytosis. Moreover, the addition of L17E to CCMV particles loaded with siRNA targeting an oncogene was shown to increase gene silencing efficacy. In an exemplary embodiment, the L17E peptide has the amino acid sequence IWLTALKFLGKHAAKHEAKQQLSKL (SEQ ID NO: 4).

In additional embodiments, the CPPs can include polypeptides having a basic amino acid rich region covalently linked to the inhibiting peptide. As used herein, the term “basic amino acid rich region” relates to a region of a protein with a high content of the basic amino acids such as arginine, histidine, asparagine, glutamine, lysine. A “basic amino acid rich region” may have, for example 15% or more (up to 100%) of basic amino acids. In some instance, a “basic amino acid rich region” may have less than 15% of basic amino acids and still function as a transport agent region. More preferably, a basic amino acid region will have 30% or more (up to 100%) of basic amino acids.

The CPPs may further include a proline rich region. As used herein, the term proline rich region refers to a region of a polypeptide with 5% or more (up to 100%) of proline in its sequence. In some instance, a proline rich region may have between 5% and 15% of prolines. Additionally, a proline rich region refers to a region, of a polypeptide containing more prolines than what is generally observed in naturally occurring proteins (e.g., proteins encoded by the human genome). Proline rich regions of the present invention can function as a transport agent region.

Other CPPs that have been tested in other contexts, (i.e., to show that they work through the use of reporter sequences), are known. One transport peptide, AAVLLPVLLAAP (SEQ ID NO: 5), is rich in proline. This transport made as a GST-MTS fusion protein and is derived from the h region of the Kaposi FGF signal sequence (Royas et al. (1998) Nature Biotech. 16: 370-375). Another example is the sperm fertiline alpha peptide, HPIQIAAFLARIPPISSIGTCILK (SEQ ID NO: 6) (See Pecheur, J. Sainte-Marie, A. Bienvenuje, D. Hoekstra. 1999. J. Membrane Biol. 167: 1-17).

Additional CPPs for use in a composition described herein can include a GGRRRRRRRRR-amide (KTG Pharmaceuticals, Inc.), a cell-permeant miniature protein (CPMP) that embodies a penta-Arg motif, and an amphipathic peptide, GGACGAGGACGAGCACUUC (SEQ ID NO: 7).

In some embodiments, the plant virus particle or VLP can be non-covalently linked to a transfection agent. An example of a non-covalently linked polypeptide transfection agent is the Chariot protein delivery system (See U.S. Pat. No. 6,841,535; Morris et al. (1999) J. Biol. Chem. 274(35):24941-24946; and Morris et al. (2001) Nature Biotech. 19:1173-1176), all herein incorporated by reference in their entirety.

The Chariot protein delivery system includes a peptide transfection agent that can non-covalently complex with the surface of the therapeutic nucleic acid loaded plant virus particle or VLP. Upon cellular internalization, the transfection agent dissociates from the nanoparticle. The complex of the Chariot transfection peptide and the therapeutic nucleic acid loaded plant virus particle or VLP can be delivered to and internalized by mammalian cells allowing for higher dosages of therapeutics to be delivered to the site of pathology.

In some embodiments, the plant virus particle or VLP can include a proton sponge mechanism. A proton sponge mechanism can include cationic polymers containing nitrogen atoms that can be protonated (e.g. polyethylenimine, “PEI”) and which act as a ‘proton sponge’. A proton sponge can be used to attract the protons in the endosome, leading to diffusion of more protons accompanied by chloride ions into the endosome. When osmotic pressure becomes high enough to destroy the endosome, the nucleic acid particles are released into the cytosol.

In some embodiments, a coating can be added to the exterior of the nucleic acid loaded plant virus particle or VLP to improve bioavailability. Administering plant virus particles to a subject can sometimes generate an immune response. An “immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, invading pathogens, cells or tissues infected with pathogens, or, in cases of autoimmunity or pathological inflammation, normal human cells or tissues. Components of an immune response can be detected in vitro by various methods that are well known to those of ordinary skill in the art.

Accordingly, in some embodiments it may be preferable to modify the exterior of the plant virus particles or VLPs and/or take other steps to decrease the immune response. For example, an immunosuppressant compound can be administered to a subject concomitantly with a plant virus vector composition described herein to decrease the immune response. More preferably, a therapeutic nucleic acid-loaded plant virus particle or VLP can be modified to decrease its immunogenicity. Examples of methods suitable for decreasing immunity include attachment of anti-fouling (e.g., zwitterionic) polymers, glycosylation of the virus carrier, and PEGylation.

In some embodiments, the immunogenicity of the therapeutic nucleic acid-loaded plant virus particle or VLP is decreased by PEGylation. PEGylation is the process of covalent attachment of polyethylene glycol (PEG) polymer chains to a molecule such as a filamentous plant virus carrier. PEGylation can be achieved by incubation of a reactive derivative of PEG with the plant virus particle exterior. The covalent attachment of PEG to the nucleic acid-loaded plant virus particle or virus-like particle can “mask” the composition from the host's immune system and reduce production of antibodies against the composition. PEGylation also may provide other benefits. PEGylation can be used to vary the circulation time of the nucleic acid-loaded plant virus particle or virus-like particle.

The first step of PEGylation is providing suitable functionalization of the PEG polymer at one or both terminal positions of the polymer. The chemically active or activated derivatives of the PEG polymer are prepared to attach the PEG to the plant virus particle. There are generally two methods that can be used to carry out PEGylation; a solution phase batch process and an on-column fed-batch process. The simple and commonly adopted batch process involves the mixing of reagents together in a suitable buffer solution, preferably at a temperature between 4 and 6° C., followed by the separation and purification of the desired product using a chromatographic technique.

The plant virus vector compositions described herein can be used to introduce a therapeutic nucleic acid into a cell. The method generally involves contacting the cell with a composition including a plant virus particle loaded with a therapeutic nucleus acid, wherein the nucleic acid is taken up into the cell. Techniques known in the art can used to measure the efficiency of the plant virus vector compositions described herein to deliver therapeutic nucleic acids to a cell.

The term “cell” as used herein is intended to refer to well-characterized homogenous, biologically pure populations of cells grown in vitro. These cells may be eukaryotic cells that are neoplastic or which have been “immortalized” in vitro by methods known in the art, as well as primary cells, or prokaryotic cells. The cell line or host cell is preferably of mammalian origin, but cell lines or host cells of non-mammalian origin may be employed, including plant, insect, yeast, fungal or bacterial sources.

In some embodiments, the cell comprises stem cells, committed stem cells, differentiated cells, primary cells, and tumor cells. Examples of stem cells include, but are not limited to, embryonic stem cells, bone marrow stem cells and umbilical cord stem cells. Other examples of cells used in various embodiments include, but are not limited to, osteoblasts, myoblasts, neuroblasts, fibroblasts, glioblasts, germ cells, hepatocytes, chondrocytes, keratinocytes, smooth muscle cells, cardiac muscle cells, connective tissue cells, glial cells, epithelial cells, endothelial cells, hormone-secreting cells, cells of the immune system, and neurons.

Atypical or abnormal cells, such as tumor cells, can also be used herein. Tumor cells cultured on substrates described herein can provide more accurate representations of the native tumor environment in the body for the assessment of drug treatments. Growth of tumor cells on the substrates described herein can facilitate characterization of biochemical pathways and activities of the tumor, including gene expression, receptor expression, and polypeptide production, in an in vivo-like environment allowing for the development of drugs that specifically target the tumor.

Additional aspects relate the use of plant virus vector compositions described herein for the treatment of a disease or disorder in a subject. The method generally involves administering to the subject a therapeutically effective amount of a plant virus vector composition including a plant virus particle or virus like particle (VLP) and a therapeutic nucleic acid, wherein the nucleic acid is encapsulated within the plant virus particle or VLP. In some embodiments, the compositions described herein can facilitate the delivery of DNA or RNA therapeutic nucleic acid as therapy for genetic disease in a subject by supplying deficient or absent gene products to treat any genetic disease or by silencing gene expression.

Gene silencing has been shown to be an effective strategy in the treatment of cancer. For example, gene silencing can be used to inhibit cell proliferation and/or induce G0/G1 arrest in cancer cells. Therefore, some embodiments described herein also relate to methods of treating cancer in a subject in need thereof by administering to the subject a therapeutically effective amount of a plant virus vector composition. The composition includes a plant virus particle or VLP and a cancer therapeutic nucleic acid, such as an oncogene targeted therapeutic nucleic acid, wherein the nucleic acid is encapsulated within the plant virus particle or VLP. In some embodiments, the composition includes a plant virus particle or VLP and an oncogene targeted RNAi construct, wherein the RNAi construct is encapsulated within the plant virus particle or VLP. In some embodiments, a targeting moiety can also be attached to the RNAi construct loaded plant virus particle or VLP. In certain embodiments, the plant virus particle is an icosahedral-shaped plant virus particle. In certain embodiments, the targeting moiety can include the M-lycotoxin peptide (L17E). In some embodiments, the RNAi construct can include an oncogene targeted siRNA. In an exemplary embodiment, the oncogene targeted siRNA includes a FOXA1 oncogene targeting siRNA.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancer cell” refers to a cell undergoing early, intermediate or advanced stages of multi-step neoplastic progression. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. Cancer cells include “hyperplastic cells,” that is, cells in the early stages of malignant progression, “dysplastic cells,” that is, cells in the intermediate stages of neoplastic progression, and “neoplastic cells,” that is, cells in the advanced stages of neoplastic progression.

The cancers treated by compositions and methods described herein can include the following: leukemias, such as but not limited to, acute leukemia, acute lymphocytic leukemia, acute myelocytic leukemias, such as, myeloblastic, promyelocytic, myelomonocytic, monocytic, and erythroleukemia leukemias and myelodysplastic syndrome; chronic leukemias, such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors such as but not limited to, glioma, astrocytoma, glioblastoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including but not limited to ductal carcinoma, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer such as but not limited to pheochromocytoma and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, fallopian tube cancer, and stromal tumor; esophageal cancers such as but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers such as but not limited to hepatocellular carcinoma and hepatoblastoma; gallbladder cancers such as adenocarcinoma; cholangiocarcinomas such as but not limited to papillary, nodular, and diffuse; lung cancers such as non-small cell lung cancer, squamous cell carcinoma (epidermoid carcinoma), adenocarcinoma, large-cell carcinoma and small-cell lung cancer; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, prostatic intraepithelial neoplasia, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acral lentiginous melanoma; kidney cancers such as but not limited to renal cell carcinoma, adenocarcinoma, hypemephroma, fibrosarcoma, transitional cell cancer (renal pelvis and/or uterer); Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America).

In some embodiments, the cancer is selected from the group consisting of breast cancer and prostate cancer. In particular embodiments, the cancer is a hormone-dependent breast or prostate cancer.

In some embodiments, the subject being administered a therapeutically effective amount of a cancer therapeutic nucleic acid-loaded plant virus nanoparticle or VLP is a subject who has been identified as having cancer. As is known to those skilled in the art, there are a variety of methods of identifying (i.e., diagnosing) a subject who has cancer. For example, diagnosis of cancer can include one or more of a physical exam, laboratory tests, imaging analysis, and biopsy. After cancer is diagnosed, a variety of tests may be carried out to look for specific features' characteristic of different types and or the extent of cancer in the subject. These tests include, but are not limited to, bone scans, X-rays, immunophenotyping, flow cytometry, and fluorescence in situ hybridization testing. For example, typical methods of diagnosing breast cancer can include, but are not limited to, a physical exam, digital mammogram, breast MRI, breast ultrasound, stereotactic core and/or open tumor biopsy, as well as lab tests to determine if the tumor tissue expresses estrogen and progesterone receptors. Typical methods of diagnosing prostate cancer can include, but are not limited to, physical digital rectal examination a serum prostate-specific antigen (PSA) test, transrectal ultrasound, MRI fusion biopsy, Prostate Cancer gene 3 (PCA3) assay, PCA3 test, prostatic biopsy and histologic analysis.

In some embodiments, the cancer therapeutic nucleic acid plant virus particles or VLPs are used to target cancer cells or cancer tissue in a subject. As used herein, targeting cancer tissue includes the ability of the plant virus vector compositions described herein to reach and preferably accumulate at a site of cancer after being administered to the subject, for example, where the compositions are systemically administered to a subject. The ability of cancer therapeutic nucleic acid-loaded plant virus particles or virus-like particles to target cancer tissue is supported by the in vitro cell uptake studies carried out by the inventors. See International Patent Publication WO2013/181557, the disclosure of which is incorporated herein by reference. While not intending to be bound by theory, it appears that therapeutic nucleic acid loaded plant virus particles or VLPs are drawn to the leaky vasculature caused by the angiogenesis associated with rapid tumor growth, and this leaky vasculature encourages entry for the plant virus particles or VLPs through small pores, thereby delivering the therapeutic nucleic acid-loaded plant virus particles or VLPs to the cancer cells. As a result of this preferential accumulation, embodiments of the invention can deliver about 10%, about 20%, about 30%, about 40%, or even about 50% or more of the injected dose to tumor tissue.

In some embodiments, the administration of the cancer therapeutic nucleic acid-loaded plant virus particles or VLPs can be proximal to a tumor in the subject or directly to the tumor site to provide a high local concentration of the cancer therapeutic nucleic acid-loaded plant virus particles or VLPs thereof in the tumor microenvironment (TME). In certain embodiments, the addition of one or more endolysosomal release agents, such as a CPP or a fusogenic peptide, can allow for endolysosomal escape of the cancer therapeutic nucleic acid-loaded plant virus particles or virus-like particle thereby increasing gene silencing efficiency in cancer cells.

In some embodiments, a method of treating cancer described herein can include administering an additional therapeutic or cancer therapy to the subject. A “cancer therapeutic” or “cancer therapy”, as used herein, can include any agent or treatment regimen that is capable of negatively affecting cancer in an animal, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of an animal with cancer. Cancer therapeutics can include one or more therapies such as, but not limited to, chemotherapies, radiation therapies, hormonal therapies, and/or biological therapies/immunotherapies. A reduction, for example, in cancer volume, growth, migration, and/or dispersal in a subject may be indicative of the efficacy of a given therapy.

In some embodiments, the method can include the step of administering a therapeutically effective amount of an additional anticancer therapeutic agent to the subject. Additional anticancer therapeutic agents can be in the form of biologically active ligands, small molecules, peptides, polypeptides, proteins, DNA fragments, DNA plasmids, interfering RNA molecules, such as siRNAs, oligonucleotides, and DNA encoding for shRNA. In some embodiments, cytotoxic compounds are included in an anticancer agent described herein. Cytotoxic compounds include small-molecule drugs such as doxorubicin, methotrexate, vincristine, and pyrimidine and purine analogs, referred to herein as antitumor agents.

The additional anticancer therapeutic agent can include an anticancer or an antiproliferative agent that exerts an antineoplastic, chemotherapeutic, antiviral, antimitotic, antitumorgenic, and/or immunotherapeutic effects, e.g., prevent the development, maturation, or spread of neoplastic cells, directly on the tumor cell, e.g., by cytostatic or cytocidal effects, and not indirectly through mechanisms such as biological response modification. There are large numbers of anti-proliferative agent agents available in commercial use, in clinical evaluation and in pre-clinical development. For convenience of discussion, anti-proliferative agents are classified into the following classes, subtypes and species: ACE inhibitors, alkylating agents, angiogenesis inhibitors, angiostatin, anthracyclines/DNA intercalators, anti-cancer antibiotics or antibiotic-type agents, antimetabolites, antimetastatic compounds, asparaginases, bisphosphonates, cGMP phosphodiesterase inhibitors, calcium carbonate, cyclooxygenase-2 inhibitors, DHA derivatives, DNA topoisomerase, endostatin, epipodophylotoxins, genistein, hormonal anticancer agents, hydrophilic bile acids (URSO), immunomodulators or immunological agents, integrin antagonists, interferon antagonists or agents, MMP inhibitors, miscellaneous antineoplastic agents, monoclonal antibodies, nitrosoureas, NSAIDs, ornithine decarboxylase inhibitors, pBATTs, radio/chemo sensitizers/protectors, retinoids, selective inhibitors of proliferation and migration of endothelial cells, selenium, stromelysin inhibitors, taxanes, vaccines, and vinca alkaloids.

The major categories that some anti-proliferative agents fall into include antimetabolite agents, alkylating agents, antibiotic-type agents, hormonal anticancer agents, immunological agents, interferon-type agents, and a category of miscellaneous antineoplastic agents. Some anti-proliferative agents operate through multiple or unknown mechanisms and can thus be classified into more than one category.

Examples of anticancer therapeutic agents that can be administered in combination with a therapeutic nucleic acid-loaded plant virus particle or VLP described herein include Taxol, Adriamycin, dactinomycin, bleomycin, vinblastine, cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; temozolomide, teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Additional anticancer therapeutic agents for the treatment of prostate cancer include systemic chemotherapeutics. Typically for the treatment of prostate cancer, standard systemic chemotherapy begins with docetaxel (Docefrez, Taxotere) combined with a steroid called prednisone. Additional systemic chemotherapeutics can include cabazitaxel and mitoxantrone. For subjects identified as having metastatic hormone-sensitive prostate cancer, Abiraterone acetate and prednisone can be administered in combination.

In particular embodiments, an additional anti-prostate cancer therapeutic agent can include an androgen deprivation therapy (ADT) agent, such as but not limited to LHRH agonists, LHRH antagonists, anti-androgen agents, and combinations thereof.

Additional anticancer therapeutic agents for the treatment of hormone sensitive breast cancer can include ovarian suppression drugs such as goserelin and leuprolide, aromatase inhibitors such as anastrozole, letrozole and exemestane, selective estrogen receptor modulators (SERMs) such as tamoxifen, raloxifene and toremifene, other antiestrogen drugs such as fulvestrant.

In some embodiments, the anti-cancer therapy administered to the subject in addition to the cancer therapeutic nucleic acid-loaded plant virus nanoparticles or VLPs can include the cancer ablation therapy. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, immunotherapy, and administration of immunotoxins.

In some embodiments, ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g., IL-2, interferon's, cytokine inducers). In contrast, specific cancer immunotherapy is based on certain antigens that are preferentially or solely expressed on cancer cells or predominantly expressed by other cells in the context of malignant disease (usually in vicinity of the tumor site). Specific cancer immunotherapy can be grouped into passive and active approaches.

In passive specific cancer immunotherapy substances with specificity for certain structures related to cancer that are derived from components of the immune system are administered to the patient. The most prominent and successful approaches are treatments with humanized or mouse/human chimeric monoclonal antibodies against defined cancer associated structures (such as Trastuzumab, Rituximab, Cetuximab, Bevacizumab, Alemtuzumab). The pharmacologically active substance exerts is activity as long as a sufficient concentration is present in the body of the patient, therefore administrations have to be repeated based on pharmacokinetic and pharmacodynamic considerations.

On the other hand, active specific cancer immunotherapy aims at antigen-specific stimulation of the patient's immune system to recognize and destroy cancer cells. Active specific cancer immunotherapy therefore, in general, is a therapeutic vaccination approach. There are many types of cancer vaccine approaches being pursued, such as vaccination with autologous or allogeneic whole tumor cells (in most cases genetically modified for better immune recognition), tumor cell lysates, whole tumor associated antigens (produced by means of genetic engineering or by chemical synthesis), peptides derived from protein antigens, DNA vaccines encoding for tumor associated antigens, surrogates of tumor antigens such as anti-idiotypic antibodies used as vaccine antigens, and the like. These manifold approaches are usually administered together with appropriate vaccine adjuvants and other immunomodulators in order to elicit a quantitatively and qualitatively sufficient immune response (many novel vaccine adjuvant approaches are being pursued in parallel with the development of cancer vaccines). Another set of cancer vaccine approaches relies on manipulating dendritic cells (DC) as the most important antigen presenting cell of the immune system. For example, loading with tumor antigens or tumor cell lysates, transfection with genes encoding for tumor antigens and in vivo targeting are suitable immunotherapies that can be used together with the cancer therapeutic nucleic acid-loaded plant virus particle or VLP described herein for cancer treatment.

In some embodiments, ablating the cancer includes administering a therapeutically effective amount of radiotherapy (RT) to the subject. In some embodiments, RT is administered prior to administration of the cancer therapeutic nucleic acid-loaded plant virus particles or VLPs. In some embodiments, administering to the cancer, (e.g., at a tumor site) a therapeutically effective amount of a RNAi-construct-loaded plant virus particles or virus-like particles to the subject in combination with administering radiotherapy to the subject can result in an increase in tumor infiltrating lymphocytes (TILs), such as tumor infiltrating neutrophils (TINs) at the tumor site of the subject.

Radiotherapy uses high-energy rays to treat disease, usually x-rays and similar rays (such as electrons). Radiotherapy administered to a subject can include both external and internal. External radiotherapy (or external beam radiation) aims high-energy x-rays at the tumor site including in some cases the peri-tumor margin. External radiotherapy typically includes the use of a linear accelerator (e.g., a Varian 2100C linear accelerator). External radiation therapy can include three-dimensional conformal radiation therapy (3D-CRT), image guided radiation therapy (IGRT), intensity modulated radiation therapy (IMRT), helical-tomotherapy, photon beam radiation therapy, proton beam radiation therapy, stereotactic radiosurgery and/or sterotactic body radiation therapy (SBRT).

Internal radiotherapy (brachytherapy) involves having radioactive material placed inside the body and allows a higher dose of radiation in a smaller area than might be possible with external radiation treatment. It uses a radiation source that is usually sealed in an implant. Exemplary implants include pellets, seeds, ribbons, wires, needles, capsules, balloons, or tubes. Implants are placed in your body, very close to or inside the tumor. Internal radiotherapy can include intracavitary or interstitial radiation. During intracavitary radiation, the radioactive source is placed in a body cavity (space), such as the uterus. With interstitial radiation, the implants are placed in or near the tumor, but not in a body cavity.

In some embodiments, a checkpoint inhibitor can be further administered to eradicate suppressive regulatory T cells prior to RT. Exemplary checkpoint inhibitors can include CTLA4 and PD-1/PDL-1 inhibitors. The cytotoxic T-lymphocyte—associated antigen 4 (CTLA-4) and programmed death 1 (PD-1) immune checkpoints are negative regulators of T-cell immune function and inhibition of these targets, results in increased activation of the immune system. Therefore, in some embodiments, a checkpoint inhibitor administered to a subject can include a CTLA-4 and/or PD-1 inhibitor. For example, Ipilimumab, an inhibitor of CTLA-4, is approved for the treatment of advanced or unresectable melanoma. Nivolumab and pembrolizumab, both PD-1 inhibitors, are approved to treat patients with advanced or metastatic melanoma and patients with metastatic, refractory non-small cell lung cancer. In addition, the combination of ipilimumab and nivolumab has been approved in patients with BRAF WT metastatic or unresectable melanoma.

It has been shown that moderate magnetic nanoparticle hyperthermia (mNPH) treatment administered to a tumor can generate immune-based systemic resistance to tumor rechallenge. Therefore, in some embodiments, a therapeutically effective amount of a moderate magnetic nanoparticle hyperthermia (mNPH) treatment can be administered to the subject in combination with an RNAi-construct-loaded plant virus particles or virus-like particles and radiotherapy, wherein the mNPH is activated with an alternating magnetic field (AMF) to produce moderate heat. Without being bound by theory, it is believed that plant virus-like particle immune adjuvants, such as a plant virus nanoparticle and/or a mNPH, will combine with RT-induced generation of immunogenic cell death (ICD) to expand the tumor specific effector T cell population causing longer local and distant tumor remission.

A mNPH treatment can include the use of a magnetic iron oxide nanoparticle (IONP). Once administered to the subject intratumorally, the mNPH can, in some embodiments, be activated with an alternating magnetic field (AMF) to produce moderate heat (e.g., 43°/60° min) at the tumor site. In some embodiments, the RT is hypofractionated RT (HFRT) that delivers larger but fewer doses/fractions than typical RT therapies.

When used in vivo, the cancer therapeutic nucleic acid-loaded plant virus particle or VLPs and/or additional anti-cancer therapeutic agents described herein can be administered as a pharmaceutical composition, comprising a mixture, and a pharmaceutically acceptable carrier. The cancer therapeutic nucleic acid-loaded plant virus particles or VLPs may be present in a pharmaceutical composition in an amount from 0.001 to 99.9 wt %, more preferably from about 0.01 to 99 wt %, and even more preferably from 0.1 to 95 wt %.

The cancer therapeutic nucleic acid-loaded plant virus particle or VLP, or pharmaceutical compositions comprising these particles and/or additional anti-cancer agent, may be administered by any method designed to provide the desired effect. Administration may occur enterally or parenterally; for example orally, rectally, intracisternally, intravaginally, intraperitoneally or locally. Parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, intraperitoneal injection, intracranial and intrathecal administration for CNS tumors, and direct application to the target area, for example by a catheter or other placement device.

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

The pharmaceutical compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

Suitable pharmaceutically acceptable carriers may contain inert ingredients which do not unduly inhibit the biological activity of the compounds. The pharmaceutically acceptable carriers should be biocompatible, e.g., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

A pharmaceutically acceptable carrier for a pharmaceutical composition can also include delivery systems known to the art for entraining or encapsulating drugs, such as anticancer drugs. In some embodiments, the disclosed compounds can be employed with such delivery systems including, for example, liposomes, nanoparticles, nanospheres, nanodiscs, dendrimers, and the like. See, for example Farokhzad, O. C., Jon, S., Khademhosseini, A., Tran, T. N., Lavan, D. A., and Langer, R. (2004). “Nanoparticle-aptamer bioconjugates: a new approach for targeting prostate cancer cells.” Cancer Res., 64, 7668-72; Dass, C. R. (2002). “Vehicles for oligonucleotide delivery to tumours.” J. Pharm. Pharmacol., 54, 3-27; Lysik, M. A., and Wu-Pong, S. (2003). “Innovations in oligonucleotide drug delivery.” J. Pharm. Sci., 92, 1559-73; Shoji, Y., and Nakashima, H. (2004). “Current status of delivery systems to improve target efficacy of oligonucleotides.” Curr. Pharm. Des., 10, 785-96; Allen, T. M., and Cullis, P. R. (2004). “Drug delivery systems: entering the mainstream.” Science, 303, 1818-22. The entire teachings of each reference cited in this paragraph are incorporated herein by reference.

Suitable doses can vary widely depending on the composition being used. A typical pharmaceutical composition for intravenous administration would be about 0.1 mg to about 10 g per subject per day. However, in other embodiments, doses from about 1 mg to about 1 g, or from about 10 mg to about 1 g can be used. Single or multiple administrations of the compositions may be administered depending on the dosage and frequency as required and tolerated by the subject. In any event, the administration regime should provide a sufficient quantity of the composition of this invention to effectively treat the subject.

Useful dosages of the additional anticancer agents, such as antimitotic agents, and anti-cancer plant virus particles can be determined by comparing their in vitro activity and the in vivo activity in animal models. Methods for extrapolation of effective dosages in mice, and other animals, to humans are known in the art; for example, see U.S. Pat. No. 4,938,949. An amount adequate to accomplish therapeutic or prophylactic treatment is defined as a therapeutically- or prophylactically-effective dose. In both prophylactic and therapeutic regimes, agents are usually administered in several dosages until an effect has been achieved. Effective doses of the additional anticancer agents and/or anti-cancer plant virus particles vary depending upon many different factors, including means of administration, target site, physiological state of the patient, whether the patient is human or an animal, other medications administered, and whether treatment is prophylactic or therapeutic.

The formulations may be conveniently presented in unit dosage form and may be prepared by any of the methods well known in the art of pharmacy. Preferably, such methods include the step of bringing the therapeutic nucleic acid-loaded plant virus particles or VLPs into association with a pharmaceutically acceptable carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect.

One skilled in the art can readily determine an effective amount of therapeutic nucleic acid loaded plant virus nanoparticles and/or additional cancer therapeutics to be administered to a given subject, by taking into account factors such as the size and weight of the subject; the extent of disease penetration; the age, health and sex of the subject; the route of administration; and whether the administration is local or systemic. Those skilled in the art may derive appropriate dosages and schedules of administration to suit the specific circumstances and needs of the subject. For example, suitable doses of the cancer therapeutic nucleic acid-loaded plant virus particles or VLPs to be administered can be estimated from the volume of cancer cells to be killed or volume of tumor to which the virus particles are being administered.

Useful dosages of the active agents can be determined by comparing their in vitro activity and the in vivo activity in animal models. Methods for extrapolation of effective dosages in mice, and other animals, to humans are known in the art. An amount adequate to accomplish therapeutic or prophylactic treatment is defined as a therapeutically- or prophylactically-effective dose. In both prophylactic and therapeutic regimes, agents are usually administered in several dosages until an effect has been achieved. Effective doses of the virus particles vary depending upon many different factors, including means of administration, target site, physiological state of the patient, whether the patient is human or an animal, characteristics of the subject, such as general health, age, sex, body weight and tolerance to drugs as well as the degree, severity and type of cancer, other medications administered, and whether treatment is prophylactic or therapeutic. The skilled artisan will be able to determine appropriate dosages depending on these and other factors using standard clinical techniques.

The methods described herein contemplate single as well as multiple administrations, given either simultaneously or over an extended period of time. A pharmaceutically acceptable composition containing the cancer therapeutic nucleic acid-loaded plant virus particles or VLPs and/or additional cancer therapeutic can be administered at regular intervals, depending on the nature and extent of the cancer's effects, and on an ongoing basis. Administration at a “regular interval,” as used herein, indicates that the therapeutically effective amount is administered periodically (as distinguished from a one-time dose). In one embodiment, the pharmaceutically acceptable composition containing the cancer therapeutic nucleic acid-loaded plant virus particles or VLPs and/or an additional cancer therapeutic is administered periodically, e.g., at a regular interval (e.g., bimonthly, monthly, biweekly, weekly, twice weekly, daily, twice a day or three times or more often a day).

The administration interval for a single individual can be fixed, or can be varied over time, depending on the needs of the individual. For example, in times of physical illness or stress, or if disease symptoms worsen, the interval between doses can be decreased.

For example, the administration of cancer therapeutic nucleic acid-loaded plant virus particles or VLPs and/or the additional therapeutic agent can take place at least once on day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively, at least once on week 1, 2, 3, 4, 5, 6, 7, 8, 9, 10, 11, 12, 13, 14, 15, 16, 17, 18, 19 or 20, or any combination thereof, using single or divided doses of every 60, 48, 36, 24, 12, 8, 6, 4, or 2 hours, or any combination thereof. Administration can take place at any time of day, for example, in the morning, the afternoon or evening. For instance, the administration can take place in the morning, e.g., between 6:00 a.m. and 12:00 noon; in the afternoon, e.g., after noon and before 6:00 p.m.; or in the evening, e.g., between 6:01 p.m. and midnight.

In some embodiments, the frequency of administration of cancer therapeutic nucleic acid-loaded plant virus particles or VLPs can pose challenging for clinical implementation. Therefore, in some embodiments, the anti-cancer virus particles administered to a subject can be formulated in a slow release formulation in order to sustain immune stimulation by maintaining a therapeutic concentration of the cancer therapeutic nucleic acid-loaded plant virus particles or VLPs, (e.g., at the site of a tumor) while alleviating the need for frequent administrations. In some embodiments, a slow release formulation can include a polymer-based hydrogel or a dendrimer.

In some embodiments, a slow-release formulation can include a cancer therapeutic nucleic acid-loaded plant virus particles or VLPs dendrimer hybrid aggregate. The dendrimer can include a positively-charged polyamidoamine (PAMAM) dendrimer, such as a medium-sized generation 3 (G3) or generation 4 (G4) PAMAM dendrimer. Depending on the specific application, the plant virus vector composition-dendrimer hybrid aggregates can vary in size and release rate of the compositions from the dendrimer when administered to a subject. In some embodiments, the therapeutic nucleic acid-loaded plant virus particle or VLP-dendrimer hybrid aggregates are formulated so that at low salt the assembly of the aggregates is triggered and while under physiologic salt concentrations disassembly and plant virus particles or VLP release is induced.

Examples have been included to more clearly describe particular embodiments of the invention. However, there are a wide variety of other embodiments within the scope of the present invention, which should not be limited to the particular examples provided herein.

### Example 1

In this Example, we describe a plant viral siRNA delivery platform. We established the application of CCMV to deliver siRNAs targeting first GFP for proof of concept and the forkhead box transcription factor (FOXA1) as a therapeutic target. To mediate cell trafficking and overcome the need for use of Lipofectamine, which has been co-delivered with plant viral capsids to promote the release of RNA cargo into the cytoplasm of mammalian cells facilitating protein expression, we appended CCMV with cell penetrating peptides (CPPs), specifically M-lycotoxin peptide L17E.

### Materials and Methods

### Purification and Propagation of Cowpea Chlorotic Mottle Virus (CCMV)

CCMV was propagated by mechanical inoculation using 5-10 μg of CCMV per leaf of cowpea plants, California Blackeye No. 5 (Vigna unguiculata). To isolate virus, infected leaf material was harvested 8 weeks post infection and blended with 2 mL of Buffer A (0.2 M sodium acetate buffer pH 4.8, 1 mM EDTA) per gram of tissue. The homogenate was squeezed through 3 layers of cheesecloth, collecting the liquid material. 1 volume of cold chloroform was added, mixed for 10 min and centrifuged at 15,000×g for 15 min. The supernatant was collected and precipitated by adding NaCl to a final concentration of 0.02 M and 8% PEG8000. The mixture was stirred overnight at 4° C., followed by centrifugation at 15,000×g for 10 minutes. The supernatant was discarded and the pellet was resuspended in 20 ml Buffer B (0.1 M sodium acetate buffer pH 4.8, 1 mM EDTA) by stirring for 1 h at 4° C., then centrifuged at 8000×g for 10 minutes. The supernatant was collected and centrifuged over a 20% sucrose cushion at 148,000×g for 2 hours. The pellet containing purified virus was then resuspended in 1 ml Buffer B. The concentration of the CCMV was determined at A260 and £=5.87 μL μg⁻¹ cm⁻¹.

### Disassembly CCMV Particles to Obtain Coat Proteins

To disassemble CCMV to get coat proteins, virions were dialyzed using a 3.5K MWCO Slide-a-Lyzer dialysis cassette (Thermo Scientific) in disassembly buffer (0.5 M CaCl₂, 50 mM Tris-HCl, pH 7.5, 1 mM EDTA, 1 mM DTT, 0.5 mM PMSF) at 4° C. for 24 hours. Following dialysis, the solution was centrifuged at 12,000×g for 30 minutes at 4° C. to pellet the viral RNA. The supernatant was then centrifuged at 220,000×g for 2 hours at 4° C. to pellet any non-disassociated virus particles. The supernatant containing coat proteins was then dialyzed using a 3.5K MWCO Slide-a-Lyzer dialysis cassette in protein buffer (1 M NaCl, 20 mM Tris pH 7.2, 1 mM EDTA, 1 mM DTT, 1 mM PMSF) for 24 hours and stored at 4° C. The concentration of the CCMV coat proteins was determined at A280 and ε=1.27 μl μg⁻¹ cm⁻¹.

Recombinant Production and Purification of CCMV Coat Proteins in E. coli

The CCMV coat protein (573 bp) was cloned into the vector pET28a(+) (Novagen) via NdeI and BamHI. The pET281/CCMV-CP construct was transformed into the E. coli strain ClearColi BL21(DE3) (Lucigen). 2 mL of an overnight culture was transferred to 400 mL of LB-Miller broth with 50 mg L⁻¹ kanamycin and grown at 37° C. until OD600 reached 0.6-0.8. Protein expression was induced using 0.5 mM IPTG and the culture was allowed to grow at 22° C. for 16 hours. The cultures were then placed on ice for 10 minutes and cells were harvested by centrifugation at 15,000×g for 20 min at 4° C. The supernatant was discarded and the pellet was resuspended in 20 mL bacteria lysis buffer (GoldBio) and incubated on ice for 5 minutes. Lysozyme was then added at a final concentration of 1 mg mL⁻¹ and the cell suspension was incubated at 37° C. for 1 hour. PMSF was then added at a final concentration at 1 mM and the solution was sonicated on a Q500 Sonicator (QSonic) for 15 minutes using 5 second pulses at an intensity of 40%. The solution was centrifuged at 15,000×g for 30 min at 4° C. and the supernatant was passed through a 0.45 μm filter. The cell suspension was loaded through a HisPur Cobalt Chromatography Cartridge (Thermo Scientific) and His-tagged CCMV coat proteins were collected through affinity purification as per manufacturer's protocol. 1.5 mL elutions were collected from the column using elution buffer (50 mM sodium phosphate, 300 mM sodium chloride, 250 mM imidazole; pH 7.4). Elution fractions were measured using UV-visible spectroscopy to verify that proteins were present in the fractions, and fractions were pooled accordingly. Pooled fractions of CCMV coat proteins were then dialyzed in protein buffer (1 M NaCl, 20 mM Tris pH 7.2, 1 mM EDTA, 1 mM DTT, 1 mM PMSF) for 24 hours at 4° C. using a 3.5K MWCO Slide-a-Lyzer dialysis cassette (Thermo Scientific). Dialyzed coat proteins were stored at 4° C.

### Reassembly of CCMV

For reassembly, coat proteins subunits and the desired dicer substrate siRNA (IDT) were mixed in a 6:1 (w/w) ratio in protein buffer. The mixture was dialyzed in a 7K MWCO Slide-a-Lyzer dialysis cassette (Thermo Scientific) against RNA assembly buffer (50 mM Tris pH 7.2, 50 mM NaCl, 10 mM KCl, 5 mM MgCl₂, 1 mM DTT) for at least 6 hours at 4° C., then immediately dialyzed against virus suspension buffer (50 mM sodium acetate buffer pH 4.5, 8 mM magnesium acetate) for at least 6 hours at 4° C. The assembly was purified by centrifugation through a 100 k Amicon Ultra-0.5 mL centrifugal filter (EMD Millipore) at 3000×g for 5 minutes, followed by 3 washes with virus suspension buffer.

### Transmission Electron Microscopy

CCMV samples were diluted to 0.5-0.8 mg ml⁻¹ in water and 20 μL was applied to glow-discharged carbon-coated 200 mesh grids (Electron Microscopy Sciences) for 2 minutes. Excess sample was blotted from the grids with Whatman Grade 1 filter paper, and the grids were rinsed twice with distilled water before staining with 2% (w/v) uranyl acetate for 2 minutes. Grids were imaged on a FEI Tecnai Spirit T12 transmission electron microscope operated at 200 kV.

### Chemical Labelling of CCMV

Sulfo-Cy5 NHS ester (Lumiprobe) was conjugated to CCMV through NHS chemistry to the exterior surface lysines. The reaction was performed using a 100 molar excess of dye with CCMV in 0.1 M HEPES pH 7.0, 5 mM MgCl₂ buffer containing 10% (v/v) DMSO. The reaction was allowed to proceed overnight at room temperature with gentle agitation. The reaction was purified with ultracentrifugation at 150,000×g for 1 hour over a 30% (w/v) sucrose cushion.

The peptide m-lycotoxin was conjugated to CCMV via an SM(PEG)₄ crosslinker (Thermo Scientific) through NHS chemistry to the exterior surface lysines. The reaction was performed using a 600 molar excess of SM(PEG)₄ with CCMV in 0.1 M HEPES pH 7.0, 5 mM MgCl₂ buffer for 2 hours at room temperature with gentle agitation. Excess SM(PEG)₄ was removed via centrifugation through a PD MidiTrap G-25 Sample Preparation Column (GE Healthcare). M-lycotoxin was reacted to CCMV-SM(PEG)₄ at 600 molar excess with gentle agitation over night at room temperature. The reaction was purified with ultracentrifugation at 150,000×g for 1 hour over a 30% (w/v) sucrose cushion.

### Gel Electrophoresis

For denaturing gel electrophoresis, samples were denatured by heating at 100° C. for 10 minutes in NuPage 4×LDS Sample loading buffer (Thermo Scientific). CCMV (10 μg) were loaded on 12% NuPage Bis-Tris protein gels (Thermo Scientific) and run in 1×MOPs buffer at 200V for 35 minutes. Gels were stained with Coomassie Blue. For native gel electrophoresis, 10 μg of sample was loaded into 0.8% (w/v) TAE agarose gels with 1×GelRed (Biotium) in 1×TAE buffer and run at 90V for 40 minutes. All gels were imaged on an AlphaImager HP (Protein Simple) and analyzed with Fiji.

### Cell Culture

HeLa and MCF-7 cells were obtained from the ATCC (Manassas, Va.). HeLa/GFP cells were obtained from Cell Biolabs, Inc (San Diego, Calif.). Cells were grown and maintained in Dulbecco's Modified Eagle's medium (DMEM, Cellgro) supplemented with 10% (v/v) fetal bovine serum (Atlanta Biologicals) and 1% (v/v) penicillin/streptomycin (Gibco). Cells were grown at 37° C. in a 5% CO₂ humidified incubator.

### Flow Cytometry

HeLa cells were collected using enzyme-free Hank's cell dissociation buffer (Gibco) and resuspended to 2.5×10⁶ cells mL⁻¹. Cells (5×10⁵ cells in 0.2 mL) were added to 96-well V-bottom plates (Corning 3897). CCMV particles (1×10⁵ particles per cell) were added in triplicate and incubated for 6 hours at 37° C. in a 5% CO₂ humidified incubator. Following incubation, half of the cells were centrifuged at 500×g and resuspended in DPBS with 1 mg mL⁻¹ pronase (Sigma-Aldrich) and treated at room temperature for 15 minutes. All cells were then centrifuged at 500×g and washed with FACS buffer (1 mM EDTA, 1% (v/v) FBS, 25 mM HEPES pH 7.0 in PBS) twice and fixed in 2% (v/v) paraformaldehyde in FACS buffer for 10 minutes. After fixing, cells were further washed in FACS buffer twice. Following washing, cells were resuspended in PBS and analyzed on a BD LSRII instrument. At least 10,000 gated events were recorded and data were analyzed using FlowJo 10.2 software.

### Confocal Microscopy

HeLa or HeLa/GFP cells were seeded on circular coverslips in a 24 well suspension plate (25,000 cells in 0.5 mL). Cells were allowed to grow for 24 hours at 37° C. in a 5% CO₂ humidified incubator before 1×10⁷ particles per cell were added. Following incubation with particles for 24 hours, cells were washed three times with DPBS, then fixed in 5% (v/v) paraformaldehyde, 0.3% (v/v) glutaraldehyde in DPBS for 10 minutes. Cells were then washed three times with DPBS. Cellular components were stained as follows: (A) for HeLa cells: (i) cell membranes were stained with wheat germ agglutinin, Alexa Fluor 555 conjugate (WGA-555; Invitrogen), 1:1000 in 5% (v/v) goat serum in DPBS; (ii) lysosomes were stained with an Alexa Fluor 488 anti-human LAMP-1 antibody (BioLegend), 1:500 in 5% (v/v) goat serum in DPBS); (iii) nuclei were stained with DAPI found in the mounting medium. (B) For HeLa/GFP cells: (i) cell membranes were stained with wheat germ agglutinin, Alexa Fluor 555 conjugate (WGA-555; Invitrogen), 1:1000 in 5% (v/v) goat serum in DPBS; (ii) nuclei were stained with DAPI found in the mounting medium; (iii) CCMV particles were stained with rabbit anti-CCMV antibody, 1:200 in 5% (v/v) goat serum in DPBS for 1 hour followed by goat anti-rabbit secondary antibody tagged with Alexa Fluor 647, 1:500 in 5% (v/v) goat serum in DPBS. Cells were first stained with WGA-555, then permeabilized with 0.2% (v/v) Triton X-100 for 2 minutes and then blocked with 10% (v/v) goat serum in DPBS for 1 hour. Cells were washed three times with DPBS in between treatments. Following all staining, coverslips were mounted onto slides with Fluoroshied with DAPI (Sigma-Aldrich) histology mounting medium and sealed with clear nail polish. Confocal images were obtained on a Leica TCS SPE confocal microscope with a 63× oil immersion objective. Images were analyzed with Fiji.

Cell Treatment with siRNA and Quantitative Real-Time PCR

HeLa/GFP or MCF-7 cells were plated in a 24-well treated plate at 62,500 cells mL⁻¹. 10,000,000 particles per cell of CCMV/CCM-siRNA was added to cells and incubated for 24 hours at 37° C. in a 5% CO₂ humidified incubator. Treatments were performed in triplicate. For RNA extraction, cell media was removed and 0.5 mL of TRI-Reagent (Sigma-Aldrich) was added to the cells. Cell lysate was passed several times through a pipette to form a homogenous lysate. RNA was extracted using TRI-Reagent as per manufacturer's protocol. 1 μg of RNA was used to make cDNA using the iScript gDNA Clear cDNA synthesis kit (Bio-Rad). cDNA was diluted 1:10 and 2 μL was used in a 20 μL qPCR reaction containing 1×SsoAdvanced Universal SYBR Green Supermix (Bio-Rad) and 250 nM each of forward and reverse primer (IDT). qPCR was performed using a CFX-96 touch machine (Bio-Rad) with the following parameters: 95° C. for 30 seconds, then 40 cycles of 98° C. for 10 seconds, 15 seconds at 60° C., followed by a melting curve. Data was analyzed with CFX Maestro software (Bio-Rad).

### Statistical Analysis

Results are presented as means ± the standard deviation (SD). Statistical comparisons between groups were performed using a one-way ANOVA followed by the appropriate post hoc tests. Significance was accepted at p values <0.05.

| dicer substrate | | |
|---|---|---|
| siRNA | | sequence |
| eGFP | sense | 5′ AACGAGAAGCGCGAUCACAUGGUC |
| siRNA | | C3′ (SEQ ID NO: 9) |
| | anti-sense | 5′ GGACCAUGUGAUCGCGCUUCUCGU |
| | | UGG 3′ (SEQ ID NO: 10) |
| FOXA1 | sense | 5′ GAGAGAAAAAAUCAACAGCAAACA |
| siRNA | | A3′ (SEQ ID NO: 11) |
| | anti-sense | 5′ UUGUUUGCUGUUGAUUUUUUCUCU |
| | | CUU 3′ (SEQ ID NO: 12) |
| Primer | | |
| eGFP | forward | 5′ GAACCGCATCGAGCTGAA 3′ |
| | | (SEQ ID NO: 13) |
| | reverse | 5′ TGCTTGTCGGCCATGATATAG 3′ |
| | | (SEQ ID NO: 14) |
| FOXA1 | forward | 5′ GGGGGTTTGTCTGGCATAGC 3′ |
| | | (SEQ ID NO: 15) |
| | reverse | 5′ GCACTGGGGGAAAGGTTGTG 3′ |
| | | (SEQ ID NO: 16) |
| ACTB | forward | 5′ AGGGTGAGGATGCCTCTCTT 3′ |
| | | (SEQ ID NO: 17) |
| | reverse | 5′ GGCATGGGTCAGAAGGATT 3′ |
| | | (SEQ ID NO: 18) |

Dicer Substrate siRNA

eGFP siRNA
Sense
(SEQ ID NO: 19)
5′ AACGAGAAGCGCGAUCACAUGGUCC 3′
Antisense
(SEQ ID NO: 20)
5′ GGACCAUGUGAUCGCGCUUCUCGUUGG 3′
FOXA1 siRNA
Sense
(SEQ ID NO: 21)
5′ GAGAGAAAAAAUCAACAGCAAACAA 3′
Antisense
(SEQ ID NO: 22)
5′ UUGUUUGCUGUUGAUUUUUUCUCUCUU 3′

NEGATIVE CONTROL siRNA
IDT Negative Control DsiRNA NS1
Cy3-LABELED siRNA
eGFP-Cy3 siRNA (GE Dharmacon)

PRIMERS:
eGFP-F
(SEQ ID NO: 23)
5' GAACCGCATCGAGCTGAA 3'
eGFP-R
(SEQ ID NO: 24)
5' TGCTTGTCGGCCATGATATAG 3'
FOXA1-F
(SEQ ID NO: 25)
5' GGGGGTTTGTCTGGCATAGC 3'
FOXA1-R
(SEQ ID NO: 26)
5' GCACTGGGGGAAAGGTTGTG 3'
ACTB-F
(SEQ ID NO: 27)
5' AGGGTGAGGATGCCTCTCTT 3'
ACTB-R
(SEQ ID NO: 28)
5' GGCATGGGTCAGAAGGATT 3'
CCMV CP sequence:
(SEQ ID NO: 29)
ATGTCTACAGTCGGAACAGGGAAGTTAACTCGTGCACAACGAAGGGCTGCG
GCCCGTAAGAACAAGCGGAACACTCGTGTGGTCCAACCTGTTATTGTAGAA
CCCATCGCTTCAGGCCAAGGCAAGGCTATTAAAGCATGGACCGGTTACAGC
GTATCGAAGTGGACCGCCTCTTGTGCGGCTGCCGAAGCTAAAGTAACCTCG
GCTATAACTATCTCTCTCCCTAATGAGCTATCGTCCGAAAGGAACAAGCAG
CTCAAGGTAGGTAGAGTTTTATTATGGCTTGGGTTGCTTCCCAGTGTTAGT
GGCACAGTGAAATCCTGTGTTACAGAGACGCAGACTACTGCTGCTGCCTCC
TTTCAGGTGGCATTAGCTGTGGCCGACAACTCGAAAGATGTTGTCGCTGCT
ATGTACCCCGAGGCGTTTAAGGGTATAACCCTTGAACAACTCGCCGCGGAT
TTAACGATCTACTTGTACAGCAGTGCGGCTCTCACTGAGGGCGACGTCATC
GTGCATTTGGAGGTTGAGCATGTCAGACCTACGTTTGACGACTCTTTCACT
CCGGTGTATTAG

### Results

CCMV particles were produced in black-eyes peas No. 5 by mechanical inoculation and purification from homogenized leaves by chloroform extraction, PEG precipitation, and ultracentrifugation; as an alternative, we also produced CCMV coat proteins using an E. coli expression system. First, we established whether CCMV would enter mammalian cells using HeLa cells, a well-established cancer cell line. For imaging and flow cytometry analysis, Cyanine5 (Cy5)-labeled CCMV was obtained using an NHS-activated Cy5 enabling coupling to CCMV's surface lysines (Supplementary Information). UV/vis spectroscopy indicated that CCMV was labeled with approximately 60 Cy5 dyes per CCMV particle.

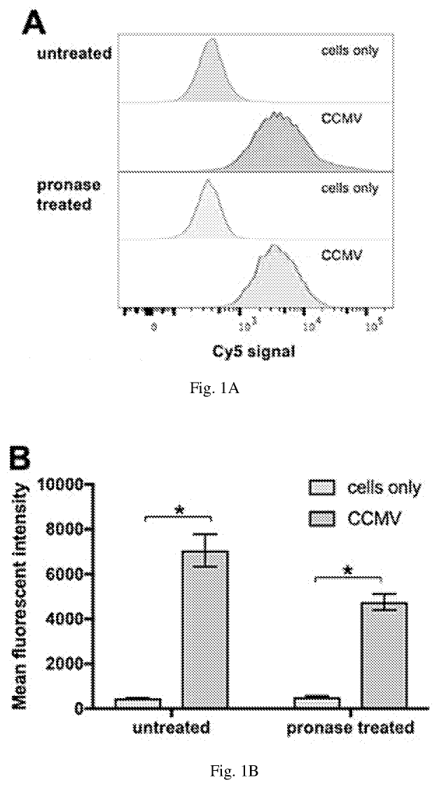

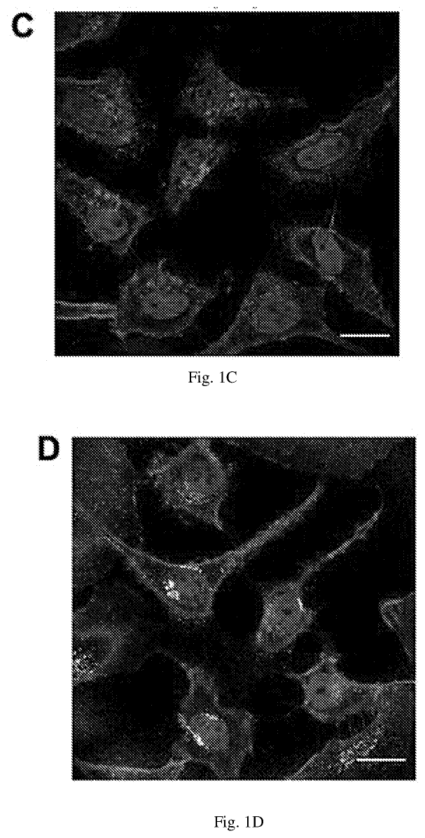

For quantitative flow cytometry assays, 1×10⁵ CCMV per cell were added and particles were allowed to interact with HeLa cells for 6 hours. Cells were treated with pronase to assess the level of surface-bound CCMV. Similarly, confocal microscopy studies were performed; flow cytometry and imaging data are in agreement and indicate that CCMV indeed enters HeLa cells; only a fraction of particles remain surface bound and hence are removed by the pronase treatment (FIGS. 1A-B). Significant co-localization with cell surface marker wheat germ agglutinin was not observed; however, staining with an endolyosomal marker (Lamp-1) revealed that CCMV is partially entrapped within endolysosomal vesicles (Manders coefficient of MCCMV vs. LAMP-1=0.32, FIGS. 1C-D); i.e. data suggest that CCMV at least partially escapes the endolyosomal compartment. Based on these encouraging data, we prepared siRNA-laden CCMV with and without CPP L17E.

siRNA encapsulation was achieved making use of pH- and salt-controlled, dis- and assembly methods; to yield CCMV loaded with siRNA, the dicer substrate siRNA as well as their non-targeted control RNAs were added at a 6:1 (w/w) ratio (FIG. 2A). Transmission electron microscopy (TEM) imaging revealed that reconstituted CCMV carrying siRNAs were structurally sound forming 30 nm-sized icosahedral particles (FIGS. 2B, C).

Next, a CPP was added; specifically, we chose the M-lycotoxin peptide L17E. This peptide was initially derived from spider venom; the L17E has Glu additions to reduce the overall positive charge and therefore enhance function. Data suggest that the L17E preferentially disrupts endolysosomal over plasma membranes; furthermore, when added to biologics (such as antibodies), L17E promotes cell uptake by micropinocytosis, thus making it a promising candidate for nanoparticle-mediated gene delivery. We reasoned that the addition of the CPP would be beneficial and increase efficacy of siRNA delivery, because our data showed that CCMV, at least in part, is entrapped in the endolysosomal compartment (see FIG. 1).

The following peptide was synthesized: IWLTALKFLGKHAAKHEAKQQLSKL (SEQ ID NO: 8) with C-terminal amide or Gly-Gly-Cys linker; the latter was used for bioconjugation to CCMV's surface lysines using an SM(PEG)4 linker (detailed protocols are listed in the Supplementary Information). Varying the peptide:CCMV ratio did not have significant impact on the labeling efficiency, SDS-PAGE revealed that ˜15-20% of CCMV's coat proteins were modified using molar ratios of 600, 900, and 1200:1 peptide:CCMV (FIG. 2D); or in other words, the conjugation yielded CCMV displaying ˜30 L17E peptides per particle (FIG. 2D). Quantification was carried out by measuring the band density comparing L17E-labeled CP vs. native CP using band analysis tool and ImageJ software. Using these methods, we then produced dual-functional CCMV loaded with siRNA and tagged with L17E peptides (FIG. 2E); SDS-PAGE revealed successful conjugation of the CPP, and agarose gel electrophoresis using a nucleic acid stain revealed successful encapsulation of the siRNA cargo (FIG. 2F). Using a fluorescently-labeled eGFP-Cy3 siRNA we determined that CCMV could encapsulated 2-3 μM siRNAs.

First, for proof-of-concept, we used GFP-expressing HeLa cells and treated these with siRNA-loaded CCMV particles with and without L17E peptide; control experiments included the use of free siRNA and CCMV-delivered siRNA in combination with lipofectamine; we used target and non-target siRNAs (at a 7.5-10 nM concentration). Confocal microscopy revealed successful gene silencing mediated by the plant viral siRNA delivery vector (FIGS. 3A-F): comparing siRNA-loaded CCMV vs. CCMV-L17E it was apparent that the addition of the L17E peptide increased efficacy; GFP expression was silenced across more cells. For either nanoparticle formulation it was apparent that GFP silencing was not achieved uniformly across all cells; however, cells that showed positive signals for CCMV (shown in in FIG. 3), loss of GFP fluorescence was apparent (FIGS. 3B-C). Quantitative analysis using real time qPCR showed that indeed addition of the L17E CPP increased the effectiveness of the gene silencing approach; while CCMV alone yielded 30% downregulation of GFP expression, the siRNA-loaded L17E-CCMV formulation achieved 50% downregulation of GFP mRNA. Interestingly, mixing CCMV with the L17E peptide did not give rise to gene silencing (sample: L17E+CCMV-siRNA). A previous study showed that physical mixtures of the L17E peptide and antibodies enabled cytosolic delivery of therapeutic antibodies. In contrast, L17E+CCMV mixtures resulted in aggregation, likely based on the polyvalent nature of the CCMV particles with its overall negative surface charge building multi-particle interlinkages with the positively-charged L17E peptide (pI˜10); therefore, preventing cell uptake, cargo delivery, and gene silencing (FIG. 3G).

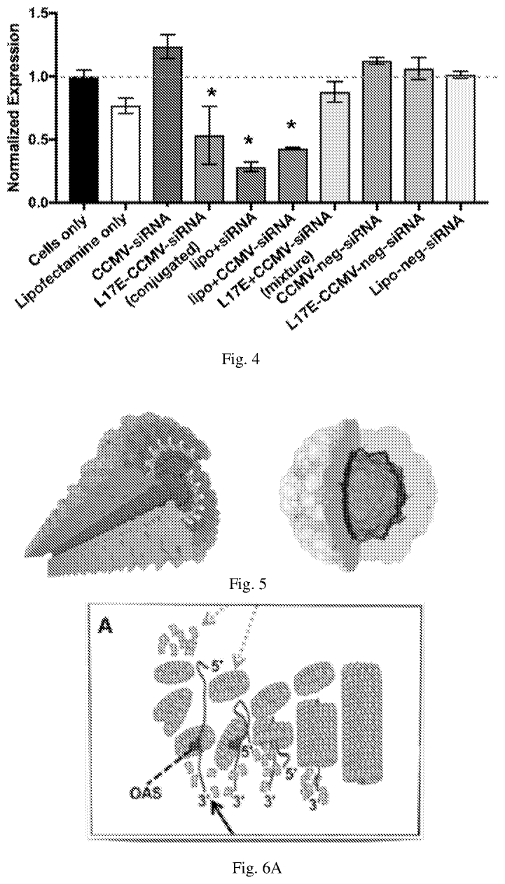

Lastly, we selected siRNAs to target FOXA1 as a potential therapeutic target in breast cancer or prostate cancer. Data indicate a critical role of FOXA1 in cell proliferation and studies suggest that gene silencing is indeed a successful strategy to inhibit cell proliferation and induce G0/G1 arrest. Here we tested whether CCMV formulated with siRNAs targeting FOXA1 would allow gene silencing using the breast cancer cell line MCF-7. Data indicate that siRNA-loaded CCMV alone was not effective in silencing the target gene FOXA1; however, conjugation of the CPP L17E restored efficacy leading to knockdown of FOXA1 mRNA levels by˜50%, matching the effectiveness of lipofectamine (FIG. 4). However, also here we found that the L17E peptide needed to be covalently conjugated and displayed on CCMV; physical mixtures of siRNA-loaded CCMV+L17E peptide had no efficacy, which again can be explained by instability of this mixture.

We demonstrate that siRNA molecules can be effectively loaded into CCMV nanoparticles, while target gene knockdown using the native CCMV protein was observed using HeLa cells overexpressing GFP, only CCMV with appended CPPs, here M-lycotoxin peptide L17E, were efficient in silencing FOXA1 gene. While plant viruses offer advantageous properties for biological applications, they have not evolved the sophisticated machinery of mammalian viral vectors, to navigate the cellular compartments of mammalian cells. Therefore, the addition of CPPs or other strategies that would prime endolyosomal escape likely will be beneficial for the development of effective plant viral gene delivery vectors. Similar observations have been made using the capsids from bacteriophages which, like plant viruses, offer a highly intriguing nanotechnology platform but lack mechanism to engage with mammalian cells. Nevertheless, gene silencing using the native CCMV capsid was apparent and imaging data indicate that the CCMV nanoparticle was only partially trapped within the endolysosomal compartment.

### Example 2

Here we describe the investigation of plant virus-based nanotechnologies for nucleic acid delivery to overcome the technological challenges that exists with contemporary viral and non-viral systems.

### Establish Efficiency

### Development and Optimization of Plant Virus-Based Vectors for Nucleic Acid Delivery

Encapsulation of Therapeutic Nucleic Acids into Plant Virus-Based Vectors. The Delivery System

Two plant virus-based nanoparticle platforms will be evaluated: 1) the high aspect ratio nanotubes formed by the nucleoprotein components of the tobacco mosaic virus (TMV, 300×18 nm) and 2) the icosahedral (or sphere-like) nanoparticles formed by the cowpea chlorotic mottle virus (CCMV, 30 nm). (FIG. 5) These platforms were chosen, because, first, feasibility for gene delivery is indicated based on literature examples: Gene delivery using CCMV has been demonstrated using self-assembled capsids encapsulating heterologous RNA; the RNA cargo was released and expressed in the cytoplasm of mammalian cells and one study indicated expression of TMV genomic RNA after cell targeting and release in the cytoplasm.

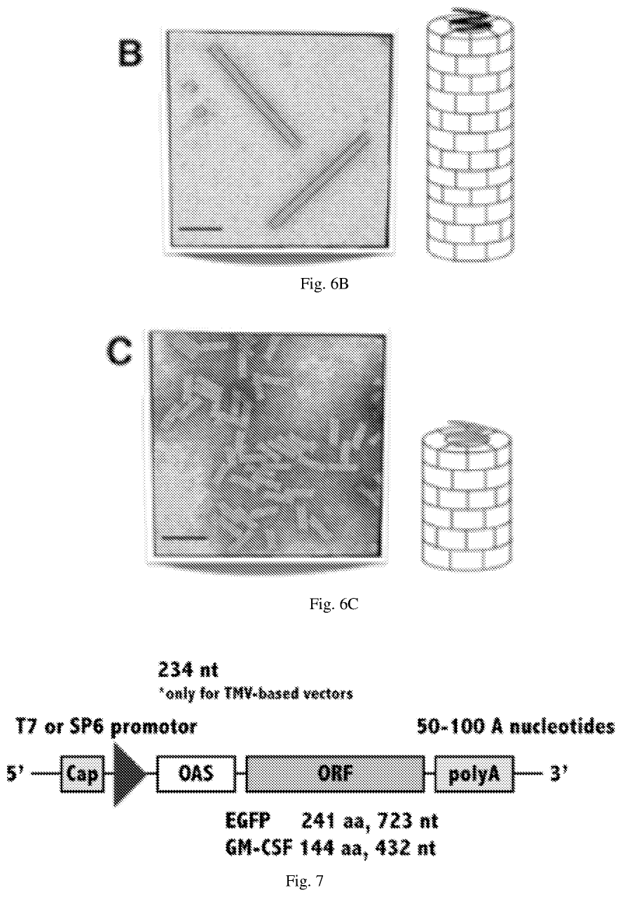

Second, the comparison between the two platforms will give insights into the design parameter nanoparticle shape: Mammalian virus-based vectors and most non-viral systems under development are spherical nanomaterials; therefore, the sphere-like platform CCMV will be a suitable model for comparison with the classical delivery systems. However, an increasing body of data suggests that elongated high aspect ratio materials have favorable properties, in particular for applications in cancer therapy, because elongated materials i) exhibit increased homing to tumor tissues (passive targeting); ii) present peptide or antibody ligands for active receptor targeting more effectively to the larger and flat vessel wall compared to their spherical counterparts; and iii) have increased immune evasion and reduced macrophage uptake, therefore rendering these platforms less immunogenic. The nanotubular structures formed by TMV offer a unique platform to investigate the structure-function relation of aspect ratio in the setting of nucleic acid therapy; while carrier shape has been investigated in classical drug delivery experiments, this design parameter is an underappreciated handle in the nucleic acid delivery field. Therefore, we will develop and evaluate TMV rods measuring 20-1000 nm in length (the diameter will be kept at 18 nm). These materials will be obtained through RNA-templated coat protein assembly; the methods are well established in our lab (FIG. 6).

### The RNA Cargo

The potential for nucleic acid therapies is wide-ranging with many different applications on the horizon. As a proof-of-concept, we will focus on the delivery of mRNA encoding reporter or therapeutic proteins. Enhanced green fluorescent protein (EGFP) will be used as a reporter protein to establish the methods. EGFP is a genetic variant of GFP with an extinction coefficient of 55,000 M-lcm-1 and a quantum yield of 0.6 and thus suited for quantitative studies assessing the gene delivery success. Then mRNA encoding granulocyte-macrophage colony-stimulating factor (GM-CSF) will be delivered to demonstrate therapeutic protein delivery. The recent approval of T-VEC, an oncolytic virus therapy encoding GM-CSF for the treatment of melanoma patients highlights the clinical utility of cytokine delivery for cancer immunotherapy. We have already demonstrated the use of plant viruses for cancer immunotherapy; the combination with GM-CSF holds potential to potentiate the therapy.

### Encapsulation

To encapsulate EGFP and GM-CSF encoding mRNAs, the following gene will be designed: The EGFP or GM-CSF open reading frame (ORF) can be obtained from the National Center for Biotechnology Information (NCBI) (gene bank entry AFA52654.1 and AAA52578.1, respectively). For encapsulation into TMV, the synthetic gene will also include the 234 nt-long origin of assembly site (OAS), which promotes assembly of TMV coat proteins yielding functional nanoparticles with encapsulated RNAs (FIG. 6). It has been shown that synthetic RNAs containing the OAS of various length and sequence are encapsulated into TMV rods with high efficiency, even complex structures, such as star- or boomerang-shaped assemblies can be obtained. The OAS will be inserted upstream of the ORF. For encapsulation into CCMV, this additional sequence is not required and therefore will not be included. To achieve efficient translation in the target cell, regulatory elements will be added: the 5′Cap structure, a 7-methyl-guanosine residue joined to the 5-end via a 5′-5′triphosphate as well as a poly(A) tail (additional regulatory elements, such as internal ribosome entry sites could also be included if deemed necessary). The polyA tail will be included in the sequence and the 5′Cap will be appended to the gene either post in vitro transcription using capping enzymes (e.g., New England Biolabs) or it is also possible to obtain capped mRNA by transcription through addition of the dinucleotide m7G(5′)-ppp-(5′)G. For in vitro transcription, the synthetic gene (FIG. 7) will be cloned into a transcription plasmid, e.g., IDT Bluescript under control of a T7 or SP6 promoter (these methods are well established in our lab, see for example FIG. 6). The plasmid will be amplified in E. coli and transcribed using available kits, e.g., MEGAscript T7 Transcription Kit (Thermo Fisher).

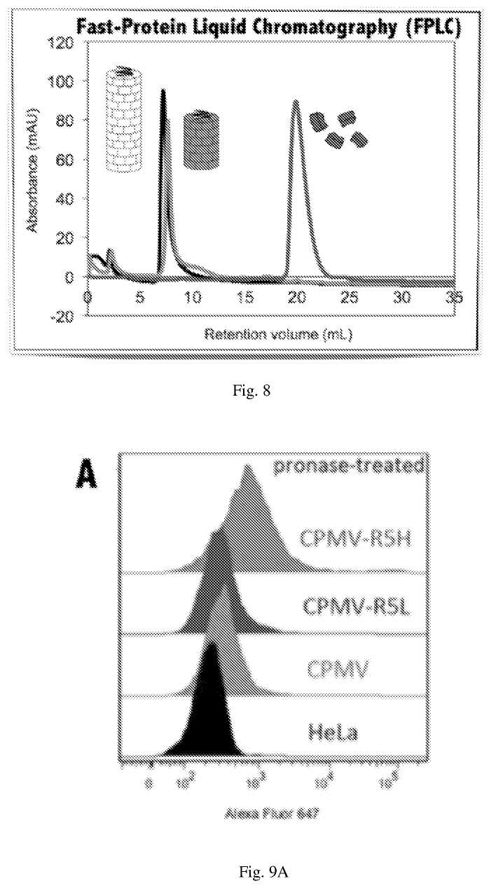

The obtained RNA transcripts will then be encapsulated into TMV and CCMV. TMV will be produced in N. benthamiana (a tobacco species) plants and CCMV will be obtained from V. unguiculate (black-eyed peas). We have access to USDA-approved greenhouse and indoor growth facilities for this research. TMV and CCMV will be purified and disassembled into their coat protein units followed by reassembly around the nucleic acid cargo of interest. To confirm structural integrity we will perform transmission electron microscopy (TEM, see FIG. 6) and fast protein liquid chromatography (FPLC, FIG. 8). Lastly protein gel electrophoresis and western blots will be used to confirm the presence of the VLP-specific coat proteins and RT-PCR and gel electrophoresis will be used to confirm and quantify RNA encapsulation (the methods are as reported).

### Aspect Ratio-Engineering

Based on length of the synthetic transcripts encoding EGFP and GM-CSF (see FIG. 7), TMV rods encapsulating a single copy of the gene are expected to measure only 20-30 nm in length (the length of the RNA defines the length of the nucleoprotein complex). To yield efficient assembly and to obtain higher aspect ratio particles, additional non-coding sequences will be added upstream of the OAS. As an alternative multiple copies of the ORF could be inserted; to enable the expression of multiple ORFs from a single mRNA translational programming elements from viruses will be included such as intervening internal ribosome entry sites or leaky stop codons.

Evaluate Plant Viral Nucleic Acid Delivery in Tissue Culture. Intracellular Trafficking

To be effective for nucleic acid delivery, cytoplasmatic cargo delivery is a key requirement. Mammalian viral vectors have evolved complex machineries that enable efficient trafficking and integration. To enable cytoplasmatic cargo delivery of nonviral vectors membrane active, pH-sensitive, fusogenic peptides can be incorporated to enable endolysosomal escape. Another strategy utilizes pH-sensitive proton polymer sponges that build up osmostic pressure within the endolysosomal compartment that eventually triggers the swelling or burst of the vesicles, resulting in cytoplasmatic cargo release.

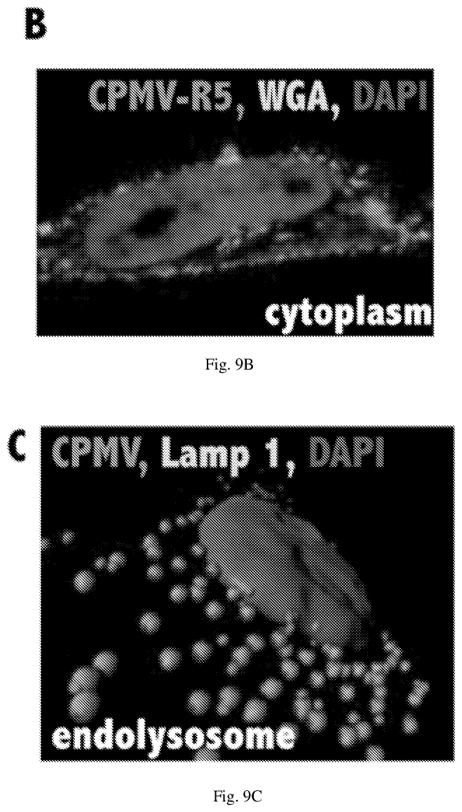

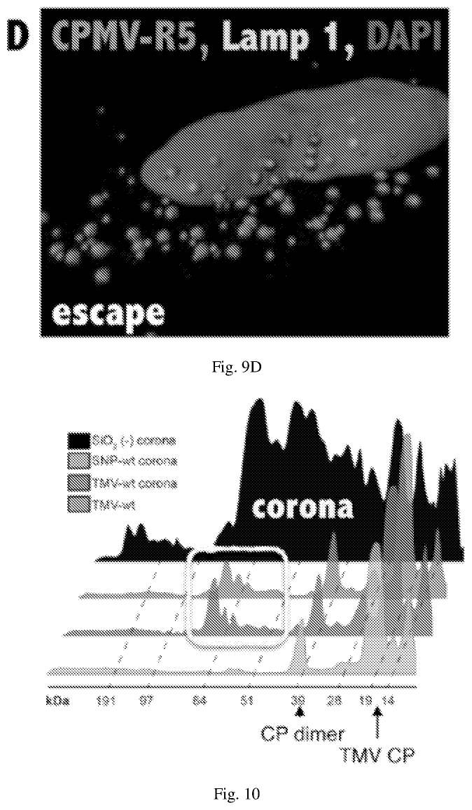

Plant viruses have not evolved machineries to integrate into the mammalian cells (which provides another layer of safety). Therefore, we will make use of surface chemistries to induce efficient transduction: we have already demonstrated the utility of fusogenic peptides conjugated to 30 nm-sized cowpea mosaic virus (CPMV) to facilitate endolysosomal escape and cytoplasmatic cargo delivery (FIG. 9). We will use nucleic-acid loaded CCMV and TMV conjugated with the fusogenic TAT motive; the sequence will either be genetically fused to the coat protein sequence of CCMV or TMV or chemically conjugated—both methods are well established in our lab. Alternative methods could also explore the use of proton sponges. Cell uptake and intracellular fates will be determined using a combination of flow cytometry and confocal imaging, as we previously described (FIG. 9). To gain a better understanding of the trafficking and release of the RNA cargo, fluorescent RNA transcripts will be produced (e.g., using RNA labeling kits, Jena Bioscience). The TAT ligand density will be optimized and we will determine any shape-dependent differences comparing CCMV and TMV of various aspect ratio. Imaging studies will be complemented with quantitative studies: cell components will be separated (e.g., using Thermo Scientific's Cytoplasmic Extraction Kit) and quantitative qRT-PCR studies will be performed to determine payload delivery to the cytoplasm.

### Transduction Efficiency

A panel of cell lines will be used to determine transduction efficiency, first EGFP, then GM-CSF will be delivered. For EGFP expression levels will be quantified based on its fluorescence using flow cytometry and fluorescence imaging; GM-CSF expression will be quantified using specific antibodies and flow cytometry as well as quantitative western blots and ELISA methods. A panel of target cancer cells will be studied: MDA-MB-231 and MCF-7 breast cancer cells, A2780 and OVCAR3 ovarian cancer cells, and SK-MEL-3, and SH-4 melanoma cells (all cell lines are available in the PI's lab and were obtained from ATCC). To determine the transduction efficiency dose dependency and time course studies will be established. Nucleic acid-loaded CCMV and TMV vectors of distinct aspect ratio will be compared side-by-side; the fusogenic TAT peptide density will be optimized based on trafficking studies; RNA transcripts with and without Cap and polyA tails will be delivered. The transduction efficiency will be determined and compared to viral and non-viral systems: as benchmark a lentiviral system will be used; the vector will be obtained from commercial sources such as Clonetech or ABM. As a model of non-viral gene delivery, we will use polyplexes of DNA and polyethylenimine (PEI); PEI is a cationic polymer that combines strong DNA compaction capacity and endolysosome-lytic activity; the methods for polyplex synthesis will be adapted from literature examples. We anticipate that these studies will illustrate the greater efficiency of the plant viral vectors compared to traditionally employed delivery vehicles.

### Establish Safety: Determine Biocompatibility in Biological Media and Immunogenicity of Plant Viral Vectors

### Stability in Biological Media

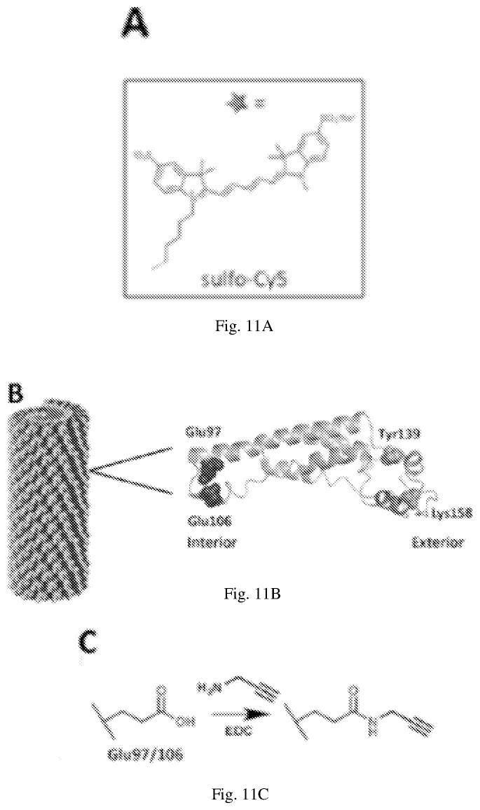

Due to their high charge density, polyplexed non-viral systems can exhibit instability in biological media and aggregate based on interactions with serum components. This is in stark contrast to the protein-based carrier, which is naturally stable in biological media. The zwitterionic nature of the protein coat renders this nanoparticle biocompatible with excellent colloidal stability and minimized interactions with plasma proteins (FIG. 10). To confirm the stability and biocompatibility of the plant viral vectors in comparison to the lentiviral vector [Clonetech or ABM] and non-viral vectors prepared from polyplexes of DNA and PEI, the nanoparticle assemblies with and without fusogenic peptides will be tested in PBS, physiological saline (0.15 M NaCl) and cell growth medium containing 10% (v/v) serum. We will measure agglomeration, precipitation, and dynamic light scattering to determine the hydrodynamic radius of the particles over time. We will also determine whether a proton corona (i.e., adsorbed plasma proteins on the carrier surface) is formed. In preliminary experiments we determined that, while plant viral vectors also exhibit a protein corona, the corona formed was significantly less abundant compared to the corona formed on synthetic nanoparticles (in this case silica nanoparticles) (FIG. 10). We will measure the degree of protein corona formation and use mass spectrometry to identify of the molecular composition of the bound proteins using established methods.

### Stealth and Camouflage

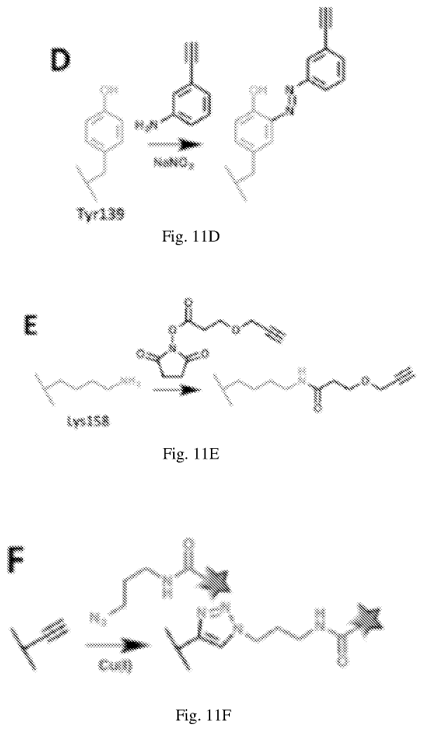

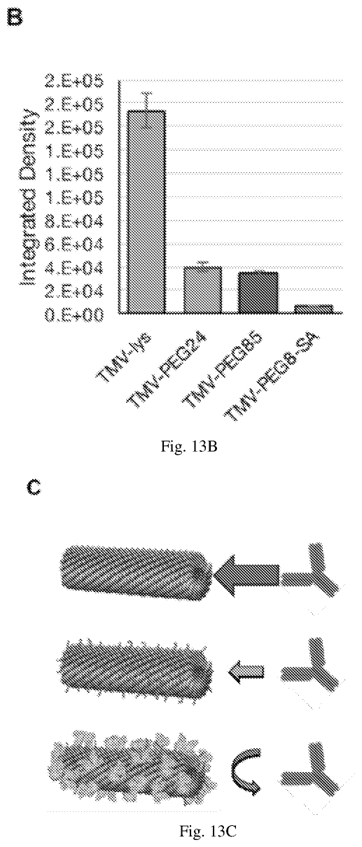

The effect of surface coatings will be evaluated with PEG and self-proteins/peptides to stealth or camouflage the plant viral nanoparticles and therefore protect from immune surveillance. We and others have shown that conjugation of PEG reduces the immunogenic properties of plant viruses and other protein drugs. Furthermore, we recently showed that coating with self-proteins such as albumin induces a camouflage and stealth effect of the plant viral vector, therefore avoiding uptake by cells of the mononuclear system and avoids recognition by antibodies (see FIG. 13). From a manufacturing point of view, the shielding through protein or peptide coatings is advantageous, because these protein or peptide sequences could be genetically introduced into the carrier this avoiding post-harvest processing. PEG-coated stealth vectors and camouflaged particles displaying albumin or CD47 peptide [GNYTCEVTELTREGETIIELK], which has been identified as the minimal self-peptide will be prepared. To screen several formulations chemical ligation strategies (FIG. 11) will be used. The ratio of stealth/camouflage vs. fusogenic peptide will be carefully optimized to provide biocompatibility while maintaining high transduction efficiency. Stability in biological media as a function of surface chemistry will be studied.

### Red Blood Cell Lysis

We have previously confirmed that TMV shows excellent blood compatibility and does not induce red blood cell (RBC) disruption (hemolysis) or blood coagulation. Here we will compare TMV and CCMV-based nucleic acid delivery vehicles side-by-side with viral and non-viral vectors to determine their blood compatibility as a function of surface chemistry (stealth vs. camouflage, and fusogenic peptides coatings in various densities and ratios). The methods will be as we previously described in Bruckman et al. (Virology 2014; 449:163-73). In brief, nanoparticle formulations will be incubated with RBCs and released hemoglobin (which is released after RBC lysis) will be determined photometrically. To assess potential aggregation and clotting rotational thromboelastometry (ROTEM) assay will be used.

### Determine Immunogenic Properties—Immune Cell Uptake and Cytokine Profiles

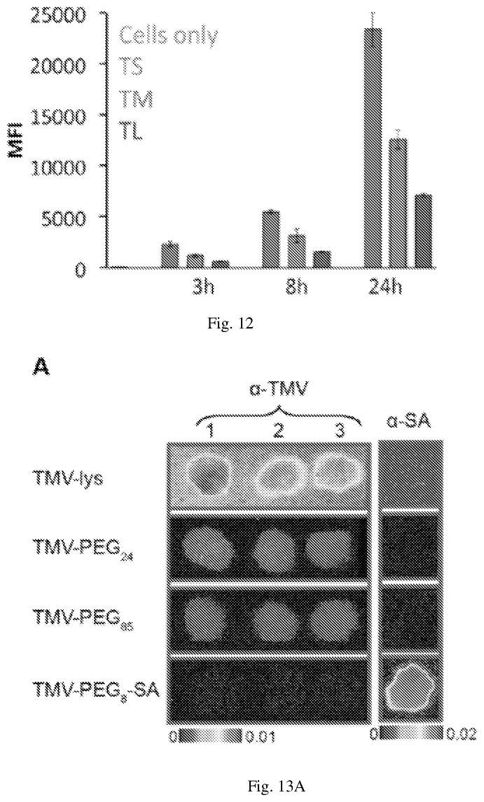

Immunogenicity is linked with the uptake and processing of nanomaterials in cells of the immune system. Therefore, we will evaluate uptake of the plant viral vectors in macrophages in dependence of particles/cell ratio and time and as a function of surface chemistry and carrier shape; specifically, we will investigate the role of aspect ratio using TMV-based carrier systems of defined length (see FIG. 6), because we have previously shown that higher aspect ratio materials avoid uptake by macrophages (FIG. 12), therefore rendering the materials less immunogenic. Uptake will be quantified and compared to other contemporary viral and non-viral systems. Native and fusogenic particles with and without PEG and self-coatings (albumin or CD47 peptide) will be studied using murine and human macrophage cell lines (J774, RAW264.7, KG-1a, SC, MD available from ATCC). Using ELISpot assays [Abcam] we will also determine potential cytokine release from these cells testing for IL4, IL6, TNFα, and IFNγ to gain insights into potential activation of the immune system. Understanding nanoparticle-macrophage interactions is expected to provide mechanistic insights into in vivo properties including their immunogenicity.

### Antibody Recognition

We will determine whether antibodies recognize and neutralize the plant viral vectors, i.e., whether antibody prevent target cell interactions, gene delivery, and transduction. First, we will use ELISA to assay whether TMV and CCMV-specific antibodies (available the PI's lab) recognize the carriers. We have shown that PEG coatings and albumin coatings significantly reduce or eliminate antibody recognition (FIG. 13). Second, we will determine whether the presence of carrier specific antibodies interferes with gene delivery, i.e., reduces the efficiency of the process. Together these studies will illustrate the safety of the plant virus-based vector compared to contemporary viral and non-viral systems.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

What is claimed is:

1. A nanoparticle comprising a plant virus or plant virus like particle (VLP), an exogenous therapeutic nucleic acid encapsulated within the plant virus or plant VLP, and one or more fusogenic peptides or cell penetrating peptides conjugated to an exterior surface of the plant virus or plant VLP.

2. The nanoparticle of claim 1, wherein the therapeutic nucleic acid is capable of treating, ameliorating, attenuating, and/or eliminating symptoms of a disease or disorder in the subject when the composition is introduced to or within a cell or tissue of the subject with a disease or disorder.

3. The nanoparticle of claim 1, wherein the therapeutic nucleic acid comprises a RNAi construct.

4. The nanoparticle of claim 1, wherein the RNAi construct is a siRNA.

5. The nanoparticle of claim 4, the siRNA comprising a siRNA targeting an oncogene.

6. The nanoparticle of claim 1, the therapeutic nucleic acid comprising a viral genome and a heterologous therapeutic gene.

7. The nanoparticle of claim 1, wherein the therapeutic nucleic acid comprises an mRNA encoding a therapeutic protein.

8. The nanoparticle of claim 1, the one or more fusogenic peptides or cell penetrating peptides comprising a L17E M-lycotoxin peptide.

9. The plant virus vector of claim 1, wherein the plant virus or VLP comprises stealth coating selected from the group consisting of PEG, albumin, and CD47 peptide.

10. A method of expressing a therapeutic nucleic acid in a mammal, comprising administering a plant virus particle composition to the mammal, the composition including a plant virus or plant virus-like particle (VLP), a therapeutic nucleic acid encapsulated within the plant virus or plant VLP, and one or more fusogenic peptides or cell penetrating peptides conjugated to an exterior surface of the plant virus or plant VLP.

11. The method of claim 10, wherein the plant virus vector is administered by intravenous injection.

12. The method of claim 10, wherein the therapeutic nucleic acid is noncovalently loaded into the plant virus particle or VLP.

13. The method of claim 10, wherein the plant virus or plant VLP is an icosahedral-shaped plant virus or plant VLP.

14. The method of claim 10, wherein the plant virus or plant VLP belongs to the Bromoviridae family.

15. The method of claim 10, wherein the plant virus or plant VLP is a cowpea chlorotic mottle virus (CCMV) or CCMV VLP.

16. The method of claim 10, wherein the therapeutic nucleic acid is capable of treating, ameliorating, attenuating, and/or eliminating symptoms of a disease or disorder in the subject when the composition is introduced to or within a cell or tissue of the subject with a disease or disorder.

17. The method of claim 10, wherein the therapeutic nucleic acid comprises a RNAi construct

18. The method of claim 17, wherein the RNAi construct is a siRNA.

19. The method of claim 18, the siRNA comprising a siRNA targeting an oncogene.

20. The method of claim 33, the one or more fusongenic peptide or cell penetrating peptide comprising a L17E M-lycotoxin peptide.


SEQUENCE LISTING (29 sequences)

SEQ ID NO: 1 - protein, 12 aa, Artificial Sequence
  Synthetic Construct
GRKKRRQRRRPQ

SEQ ID NO: 2 - protein, 21 aa, Artificial Sequence
  Synthetic Construct
KETWWETWWTEWSQPKKKRKV

SEQ ID NO: 3 - protein, 16 aa, Artificial Sequence
  Synthetic Construct
RQIKIWFQNRRMKWKK

SEQ ID NO: 4 - protein, 25 aa, Artificial Sequence
  Synhetic Construct
IWLTALKFLGKHAAKHEAKQQLSKL

SEQ ID NO: 5 - protein, 12 aa, Artificial Sequence
  Synthetic Construct
AAVLLPVLLAAP

SEQ ID NO: 6 - protein, 24 aa, Artificial Sequence
  Synthetic Construct
HPIQIAAFLARIPPISSIGTCILK

SEQ ID NO: 7 - RNA, 19 nt, Artificial Sequence
  Synthetic Construct
ggacgaggacgagcacuuc

SEQ ID NO: 8 - protein, 25 aa, Artificial Sequence
  Synthetic Construct
IWLTALKFLGKHAAKHEAKQQLSKL

SEQ ID NO: 9 - RNA, 25 nt, Artificial Sequence
  Synthetic Construct
aacgagaagcgcgaucacauggucc

SEQ ID NO: 10 - DNA, 27 bp, Artificial Sequence
  Synthetic Construct
ggaccaugugaucgcgcuucucguugg

SEQ ID NO: 11 - RNA, 25 nt, Artificial Sequence
  Synthetic Construct
gagagaaaaaaucaacagcaaacaa

SEQ ID NO: 12 - RNA, 27 nt, Artificial Sequence
  Synthetic Construct
uuguuugcuguugauuuuuucucucuu

SEQ ID NO: 13 - DNA, 18 bp, Artificial Sequence
  Synthetic Construct
gaaccgcatcgagctgaa

SEQ ID NO: 14 - DNA, 21 bp, Artificial Sequence
  Synthetic Contruct
tgcttgtcggccatgatatag

SEQ ID NO: 15 - DNA, 20 bp, Artificial Sequence
  Synthetic Construct
gggggtttgtctggcatagc

SEQ ID NO: 16 - DNA, 20 bp, Artificial Sequence
  Synthetic Construct
gcactgggggaaaggttgtg

SEQ ID NO: 17 - DNA, 20 bp, Artificial Sequence
  Synthetic Construct
agggtgaggatgcctctctt

SEQ ID NO: 18 - DNA, 19 bp, Artificial Sequence
  Synthetic Construct
ggcatgggtcagaaggatt

SEQ ID NO: 19 - RNA, 25 nt, Artificial Sequence
  Synthetic Construct
aacgagaagcgcgaucacauggucc

SEQ ID NO: 20 - RNA, 27 nt, Artificial Sequence
  Synthetic Construct
ggaccaugugaucgcgcuucucguugg

SEQ ID NO: 21 - RNA, 25 nt, Artificial Sequence
  Synthetic Construct
gagagaaaaaaucaacagcaaacaa

SEQ ID NO: 22 - RNA, 27 nt, Artificial Sequence
  Synthetic Construct
uuguuugcuguugauuuuuucucucuu

SEQ ID NO: 23 - DNA, 18 bp, Artificial Sequence
  Synthetic Construct
gaaccgcatcgagctgaa

SEQ ID NO: 24 - DNA, 21 bp, Artificial Sequence
  Synthetic Construct
tgcttgtcggccatgatatag

SEQ ID NO: 25 - DNA, 20 bp, Artificial Sequence
  Synthetic Construct
gggggtttgtctggcatagc

SEQ ID NO: 26 - DNA, 20 bp, Artificial Sequence
  Synthetic Construct
gcactgggggaaaggttgtg

SEQ ID NO: 27 - DNA, 20 bp, Artificial Sequence
  Synthetic Construct
agggtgaggatgcctctctt

SEQ ID NO: 28 - DNA, 19 bp, Artificial Sequence
  Synthetic Construct
ggcatgggtcagaaggatt

SEQ ID NO: 29 - DNA, 573 bp, Artificial Sequence
  Synthetic Construct
atgtctacagtcggaacagggaagttaactcgtgcacaacgaagggctgcggcccgtaagaacaagcggaacactcgtgtggtccaacctgttattgtagaacccatcgcttcaggccaaggcaaggctattaaagcatggaccggttacagcgtatcgaagtggaccgcctcttgtgcggctgccgaagctaaagtaacctcggctataactatctctctccctaatgagctatcgtccgaaaggaacaagcagctcaaggtaggtagagttttattatggcttgggttgcttcccagtgttagtggcacagtgaaatcctgtgttacagagacgcagactactgctgctgcctcctttcaggtggcattagctgtggccgacaactcgaaagatgttgtcgctgctatgtaccccgaggcgtttaagggtataacccttgaacaactcgccgcggatttaacgatctacttgtacagcagtgcggctctcactgagggcgacgtcatcgtgcatttggaggttgagcatgtcagacctacgtttgacgactctttcactccggtgtattag